(19) United States
(12) Patent Application Publication
(10) Pub. No.: US 2012/0184026 A1
(43) Pub. Date: Jul. 19, 2012

(54) INTEGRATED PROCESSING PLANTS

(76) Inventor: Aharon Meir Eyal, Jerusalem (IL)

(21) Appl. No.: 13/380,504

(22) PCT Filed: Dec. 21, 2011
(86) PCT No.: PCT/US11/66669

§ 371 Date: Jan. 19, 2012

Related U.S. Application Data
(60) Provisional application No. 61/473,134, filed on Apr. 7, 2011.
(60) Provisional application No. 61/483,663, filed on May 7, 2011.

(30) Foreign Application Priority Data
Dec. 21, 2010 (IL) 210161

(51) Int. Cl. C12M 1/00; B01J 19/00

(52) U.S. Cl. 435/289.1; 127/1

(57) ABSTRACT

An integrated plant comprising: (a) one or more lignocellulose processing units producing one or more sugar streams and one or more lignin streams; (b) one or more lignin-processing units processing one or more of said lignin streams into a lignin product; and (c) one or more sugar processing units processing one or more of said sugar streams into a sugar product; (d) at least one transfer mechanism transferring one or more of said sugar stream(s) to one or more of said sugar processing units over a distance of 5 km or less; and (e) at least one transfer mechanism transferring said lignin stream from one or more of said lignocellulose processing units to one or more of said lignin processing units over a distance of 5 km or less.

### RELATED APPLICATIONS

In accord with the provisions of 35 U.S.C. §119(a) and/or §365(b), this application claims priority from:

prior Israeli application IL 210161 filed Dec. 21, 2010 and entitled “A Method for Processing a Lignocellulosic Material into a Hydrolyzate Product”; which is fully incorporated herein by reference.

In addition, in accord with the provisions of 35 U.S.C. §119(e) and §363, this application claims the benefit of:

U.S. 61/473,134 Filed Apr. 7, 2011 and entitled “Lignocellulose Conversion Processes and Products”;

U.S. 61/483,663 Filed May 7, 2011 and entitled “Lignocellulose Conversion Processes and Products”;

U.S. 61/539,861 Filed Sep. 27, 2011 and entitled “A Method for Processing a Lignocellulosic Material into a Hydrolyzate Product”; and

U.S. 61/576,268 Filed Dec. 15, 2011 and entitled “Integrated Processing Plants”; each of which is fully incorporated herein by reference.

### FIELD OF THE INVENTION

This invention relates to industrial plants for processing of lignin and/or cellulose and/or sugars.

### BACKGROUND OF THE INVENTION

Industries relating to fermentation feedstock use nearly 100 million tons of carbohydrates annually to produce fuel-grade ethanol and other industrial and commercial products such as monomers for the polymer industry, e.g. lactic acid for the production of polylactide. Millions of tons of carbohydrates are also fermented every year to food and feed products, such as citric acid and lysine. Carbohydrates are attractive as an environmental-friendly substrate since they are obtained from renewable resources, presently, mainly, sucrose from sugar canes and glucose from corn and wheat starches.

An abundant and relatively-low cost carbohydrate source is woody materials, such as wood and co-products of wood processing and residues of processing agricultural products, e.g. corn stover and cobs, sugar cane bagasse and empty fruit bunches from palm oil production as well as “energy crops” that generate low-cost rapid growing biomass (e.g. switch grass).

These woody materials contain cellulose, hemicellulose and lignin as the main components and are also referred to as lignocellulose or lignocellulosic material. Such material also contains mineral salts (ashes) and organic compounds, such as tall oils.

Cellulose and hemicellulose, which together form 65-80% of lignocellulosic materials, are polysaccharides and their hydrolysis forms carbohydrates suitable for fermentation and/or chemical conversion to products of interest. Cellulose, which typically forms more than one half of the polysaccharides content, has a crystalline structure while hemicellulose does not.

### SUMMARY OF THE INVENTION

A broad aspect of the invention relates to integration of two, optionally three industrial processing units into an integrated plant. In some embodiments of the invention, integration indicates a proximity of less than 5 Kilometers.

One aspect of some embodiments of the invention relates to transfer of materials between a lignocellulose processing unit and a sugar processing unit. According to various exemplary embodiments of the invention the transfer may be in either direction. In some embodiments, sugars produced by hydrolysis and/or co-products from a lignocellulosic substrate are transferred to the sugar processing unit. Alternatively or additionally, unprocessed sugars and/or other sugar processing co-products are transferred from the sugar processing unit to the lignocellulose processing unit.

Another aspect of some embodiments of the invention relates to transfer of materials between a lignocellulose processing unit and a lignin processing unit. According to various exemplary embodiments of the invention the transfer can be in either direction. In some embodiments, lignin and/or co-products from a lignocellulosic substrate are transferred to the lignin processing unit. Alternatively or additionally, lignin products and/or other lignin processing co-products are transferred from the lignin processing unit to the lignocellulose processing unit.

Another aspect of some embodiments of the invention relates to transfer of materials between a sugar processing unit and a lignin processing unit. According to various exemplary embodiments of the invention the transfer can be in either direction. In some embodiments, lignin products and/or lignin co-products are transferred to the sugar processing unit. Alternatively or additionally, sugar products and/or sugar processing co-products are transferred from the lignin processing unit to the sugar processing unit.

An additional aspect of some embodiments of the invention relates to transfer of energy between the various unit types described above. Optionally, the energy is transferred as heat energy (e.g. in the form of steam). Alternatively or additionally, energy is transferred as electric current. In some embodiments, one or more generators provide heat from combustion of a co-product.

An additional aspect of some embodiments of the invention relates to integration of waste water treatment for waste streams various unit types described above. In some embodiments, a waste water treatment unit processes waste streams from two, or even three different unit types described above. Optionally, the waste water treatment unit is physically located in a different unit type.

It will be appreciated that the various aspects described above relate to solution of technical problems associated with transportation logistics.

Alternatively or additionally, it will be appreciated that the various aspects described above relate to solution of technical problems related to utilization or exploitation of co-products of relevant industrial processes.

In some exemplary embodiments of the invention, there is provided an integrated plant including: (a) one or more lignocellulose processing units producing one or more sugar streams and one or more lignin streams; (b) one or more lignin-processing units processing one or more of the lignin streams into a lignin product; and (c) one or more sugar processing units processing one or more of the sugar streams into a sugar product; (d) at least one transfer mechanism transferring one or more of the sugar stream(s) to one or more of the sugar processing units over a distance of 5 km or less; and (e) at least one transfer mechanism transferring the lignin stream from one or more of the lignocellulose processing units to one or more of the lignin processing units over a distance of 5 km or less. In some embodiments of the plant the lignocellulose processing units produce or more lignocellulose co-products. Alternatively or additionally, the plant include one or more second sugar processing units processing one or more second sugars into one or more second sugar products. Alternatively or additionally, the plant includes at least one energy transfer mechanism transferring energy between units in at least one relationship selected from the group consisting of: (i) from at least one lignocellulose processing unit to at least one lignin processing unit; (ii) from at least one lignocellulose processing unit to at least one sugar processing unit; (iii) from at least one sugar processing unit to at least one lignin processing unit; (iv) from at least one sugar processing unit to at least one lignocellulose processing unit; (v) from at least one lignin processing unit to at least one sugar processing unit; and (vi) from at least one lignin processing unit to at least one lignocellulose processing unit. Alternatively or additionally, the plant includes a transfer mechanism transferring a material from one or more of the lignin processing units to one or more other units. Alternatively or additionally, the plant includes a transfer mechanism transferring a material from one or more of the lignin processing units to one or more of the second sugar processing units. Alternatively or additionally, the plant includes a transfer mechanism transferring a material from at least one unit of the second sugar processing units to one or more other units. Alternatively or additionally, one or more sugar processing units in the plant is adapted to separate a sugar processing co-product from the sugar product. Alternatively or additionally, one or more of the sugar processing units in the plant includes one or more chemical sugar conversion modules adapted to chemically convert a sugar to a fermentable intermediate. Alternatively or additionally, the plant includes one or more conversion modules adapted to convert at least one sugar product produced by fermentation into a converted product. Alternatively or additionally, at least one of the one or more sugar processing units in the plant includes a recovery module adapted to recover unprocessed sugars. Alternatively or additionally, the one or more lignocellulose processing units in the plant includes one or more acid hydrolysis modules. Alternatively or additionally, the plant includes one or more waste water treatment units processing one, two or three members of the group consisting of: one or more waste streams from one or more of the lignocellulose processing units; one or more waste streams from one or more of the lignin processing units; and one or more waste streams originating from one or more of the sugar processing units. Alternatively or additionally, the plant includes one or more generators generating heat from combustion of at least one of the co-products. In some embodiments, the plant includes one or more heat transfer mechanisms transferring heat from the generator to one or more other units. Alternatively or additionally, the plant includes a transfer mechanism transferring one or more of the lignocellulose co-products to one or more other units. Alternatively or additionally, the lignocellulose co-product includes acetic acid and the plant includes an acetic acid transfer mechanism transferring acetic acid to one or more other units. Alternatively or additionally, the lignocellulose co-product includes methanol and the plant includes a methanol transfer mechanism transferring methanol to one or more other units. Alternatively or additionally, the lignocellulose co-product includes one or more tall oils and the plant includes a tall oil transfer mechanism transferring at least one of the one or more tall oils to one or more other units. Alternatively or additionally, the plant includes an acid transfer mechanism transferring acid from an acid recovery module of the one or more lignin processing units to one or more other units. Alternatively or additionally, the plant includes a solvent transfer mechanism transferring solvent from a solvent recovery module of the one or more lignin processing units to one or more other units. Alternatively or additionally, the plant includes one or more acid-recycle modules adapted to recover acid from one or more lignin processing units and return the acid to one or more other units. Alternatively or additionally, the plant includes a hydrogen transfer mechanism transferring hydrogen from one or more of the lignin processing units to one or more other units. In some embodiments, the plant produces a lignin stream including liquefied lignin. Alternatively or additionally, the one or more lignocellulose processing units process at least 10 tons of lignocellulose per hour. Alternatively or additionally, the at least one transfer mechanism transfers at least 30,000 tons of sugar/yr from the one or more lignocellulose processing units to the one or more sugar processing units.

In some exemplary embodiments of the invention, there is provided an integrated plant comprising: (a) one or more lignocellulose processing units producing one or more sugar streams, each sugar stream including one or more sugars; (b) one or more sugar processing units processing one or more sugars from one or more of the sugar streams into a sugar product; and (c) at least one transfer mechanism transferring one or more of the sugar stream(s) to one or more of the sugar processing units over a distance of 5 km or less. Alternatively or additionally, one or more of the lignocellulose processing units in the plant includes a module which produces one or more lignocellulose co-products. In some embodiments, the lignocellulose co-product includes acetic acid and the plant includes an acetic acid transfer mechanism transferring acetic acid to one or more of the sugar processing units. Alternatively or additionally, in some embodiments, the lignocellulose co-product includes methanol and the plant includes a methanol transfer mechanism transferring methanol to one or more of the sugar processing units. Alternatively or additionally, in some embodiments, the lignocellulose co-product includes one or more tall oils and the plant includes a tall oil transfer mechanism transferring at least one of the one or more tall oils to one or more of the sugar processing units. In some embodiments, of the plant, the one or more sugar processing units include one or more first sugar processing modules processing a first sugar into a first sugar product and one or more second sugar processing modules processing a second sugar into one or more second sugar products. Alternatively or additionally, the plant includes at least one transfer mechanism transferring one or more of the sugar stream(s) to one or more of the second sugar processing modules. Alternatively or additionally, the plant includes at least one transfer mechanism transferring a material from one or more of the sugar processing units to one or more of the lignocellulose processing units. Alternatively or additionally, the plant includes at least one transfer mechanism transferring a sugar product to one or more sugar product processing modules. In some embodiments, of the plant the one or more lignocellulose processing units includes one or more acid hydrolysis modules. Alternatively or additionally, the plant includes one or more waste water treatment units processing one or more waste streams from one or more of the lignocellulose processing units and/or one or more waste streams originating from one or more of the sugar processing units. Alternatively or additionally, the plant includes one or more generators generating heat from combustion of at least one of the co-products. In some embodiments, the plant includes a heat transfer mechanism transferring heat from the generator to one unit from one or more other units. Alternatively or additionally, the plant includes a transfer mechanism transferring one or more of the lignocellulose co-products to one or more of the sugar processing units. Alternatively or additionally, the plant includes a transfer mechanism transferring one or more sugar processing co-products from one or more of the sugar processing units to one or more other units. In some embodiments, the material includes one or more sugar processing co-products. Alternatively or additionally, the material includes one or more sugar processing products. Alternatively or additionally, the one or more lignocellulose processing units of the plant process at least 10 tons of lignocellulose per hour. Alternatively or additionally, the at least one transfer mechanism transfers at least 30,000 tons of sugar/yr from the one or more lignocellulose processing units to the one or more sugar processing units of the plant.

In some exemplary embodiments of the invention, there is provided an integrated plant including: (a) one or more lignocellulose processing units producing at least one lignin stream; (b) one or more lignin-processing units processing lignin from the at least one lignin stream into a lignin product; and (c) at least one transfer mechanism transferring the lignin stream from one or more of the lignocellulose processing units to one or more of the lignin processing units over a distance of 5 km or less. In some embodiments, of the plant one or more of the one or more lignocellulose processing units produce one or more co-products selected from the group consisting of tall oils, ash, resins, pitch and furfurals. Alternatively or additionally, the plant includes a transfer mechanism transferring a material from one or more of the lignin processing units to one or more of the lignocellulose processing units. Alternatively or additionally, the plant includes an acid transfer mechanism transferring acid from an acid recovery module of the one or more lignin processing units to one or more of the lignocellulose processing units. Alternatively or additionally, the plant includes a solvent transfer mechanism transferring solvent from a solvent recovery module of the one or more lignin processing units to one or more of the lignocellulose processing units. Alternatively or additionally, the plant includes one or more acid-recycle modules adapted to recover acid from one or more lignin processing units and return the acid to the one or more of the lignocellulose processing units. Alternatively or additionally, the plant includes one or more waste-water treatment units treating one or more waste streams from one or more lignocellulose processing units and/or from one or more lignin processing units. Alternatively or additionally, the plant includes a generator generating heat from combustion of at least one of the co-products. In some embodiments, the plant includes a heat transfer mechanism transferring heat from the generator from one unit to one or more other units. Alternatively or additionally, the plant includes a transfer mechanism transferring one or more of the co-products from one or more of the lignocellulose processing units to one or more other units. Alternatively or additionally, the plant includes one or more transfer mechanisms transferring a waste stream from one or more of the lignin processing units to one or more of the waste-water treatment units. Alternatively or additionally, the plant includes one or more transfer mechanisms transferring a lignin processing co-product from one or more of the lignin processing units to one or more of the lignocellulose processing units. Alternatively or additionally, the plant includes one or more transfer mechanisms transferring at least a portion of the lignin product from one or more of the lignin processing units to one or more of the lignocellulose processing units. Alternatively or additionally, the one or more lignocellulose processing units of the plant have a processing capacity of at least 10 tons of lignocellulose per hour. Alternatively or additionally, the transfer mechanism of the plant transfers at least 10,000 tons of lignin/yr from the lignocellulose processing unit(s) to the lignin-processing unit(s).

In some exemplary embodiments of the invention, there is provided an integrated plant comprising: (a) one or more lignin-processing units processing one or more lignin streams; and

(b) one or more sugar processing units processing one or more sugar streams into one or more sugar products; and (c) at least one transfer mechanism transferring one or more materials between one or more of the sugar processing units and one or more of the lignin processing units over a distance of 5 km or less in either direction. Alternatively or additionally, the plant includes one or more additional sugar processing units processing sugar into one or more additional sugar products. Alternatively or additionally, the plant includes at least one transfer mechanism transferring one or more materials between one or more of the additional sugar processing units and one or more of the lignin processing units over a distance of 5 km or less in either direction. Alternatively or additionally, the plant includes a transfer mechanism transferring a material from one or more of the sugar processing units to one or more of the additional sugar processing units. Alternatively or additionally, the plant includes a hydrogen transfer mechanism transferring hydrogen from one or more of the lignin processing units to one or more of the sugar processing units. In some embodiments of the plant, the material includes liquefied lignin. Alternatively or additionally, the plant includes a transfer mechanism transferring a material from one or more of the sugar processing units to one or more of the lignin processing units. In some embodiments, the material includes a solvent produced by an aqueous phase reforming (APR) module in one or more of the sugar processing units. In some embodiments, of the plant the material includes an alcohol. Alternatively or additionally, the plant includes a transfer mechanism transferring a material from one or more of the lignin processing units to one or more of the additional sugar processing units. In some embodiments of the plant the material includes hydrogen. Alternatively or additionally, in some embodiments of the plant the material includes liquefied lignin. Alternatively or additionally, the plant includes a transfer mechanism transferring a material from at least one unit of the sugar processing units into one or more of the lignin processing units. Alternatively or additionally, the plant includes one or more waste water treatment units processing at least one stream, optionally two streams, selected from the group consisting of: one or more waste streams from one or more of the lignin processing units; and one or more waste streams originating from one or more of the sugar processing units. Alternatively or additionally, the plant includes one or more generators generating heat from combustion of at least one of the co-products. Alternatively or additionally, the plant includes a heat transfer mechanism transferring heat from the generator to one or more other units. Alternatively or additionally, the one or more sugar processing units have a processing capacity of at least 3 tons of sugar per hour.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative, as opposed to limiting.

As used herein, the terms “comprising” and “including” or grammatical variants thereof are to be taken as specifying inclusion of the stated features, integers, actions or components without precluding the addition of one or more additional features, integers, actions, components or groups thereof.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Percentages (%) of chemicals and/or reagents and/or ingredients are W/W (weight per weight) unless otherwise indicated.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a schematic representation of an integrated plant according to various exemplary embodiments of the invention;

FIG. 2 is a schematic representation of an exemplary lignocellulose processing unit of an integrated plant according to FIG. 1 depicting exemplary modules in greater detail;

FIG. 3a is a schematic representation of an exemplary sugar processing unit of an integrated plant depicting serially arranged exemplary sugar processing modules in greater detail;

FIG. 3b is a schematic representation of an exemplary sugar processing unit of an integrated plant depicting a parallel arrangement of exemplary sugar processing modules in greater detail;

FIG. 3c is a schematic representation of an exemplary sugar processing unit of an integrated plant depicting an alternate serial arrangement of exemplary sugar processing modules in greater detail;

FIG. 4 is a schematic representation of an integrated plant with an optional waste water processing unit according to some exemplary embodiments of the invention;

FIG. 5 is a schematic representation of an exemplary lignin processing unit of an integrated plant depicting exemplary modules in greater detail;

FIG. 6 is a schematic representation of an integrated plant with optional energy transfer mechanisms depicted; and

FIG. 7 is a schematic representation of another exemplary integrated plant with multiple sugar processing units according to some embodiments of the invention.

### DETAILED DESCRIPTION OF EMBODIMENTS

Embodiments of the invention relate to integrated processing plants which include at least two unit types selected from among: lignocellulose processing units; sugar processing units and lignin processing units.

Specifically, some embodiments of the invention can be used to hydrolytically process a lignocellulosic substrate and further process resultant lignin and/or sugars into products and/or co-products. In some embodiments, there is integration of processing of lignin and/or lignin products and/or lignin co-products with sugars and/or sugar products and/or sugar co-products.

The principles and operation of integrated plants according to various exemplary embodiments of the invention may be better understood with reference to the drawings and accompanying descriptions.

Before explaining at least one embodiment of the invention in detail, it is to be understood that the invention is not limited in its application to the details set forth in the following description. The invention is capable of other embodiments or of being practiced or carried out in various ways. Also, it is to be understood that the phraseology and terminology employed herein is for the purpose of description only and that the scope of the invention is defined by the claims.

System Overview

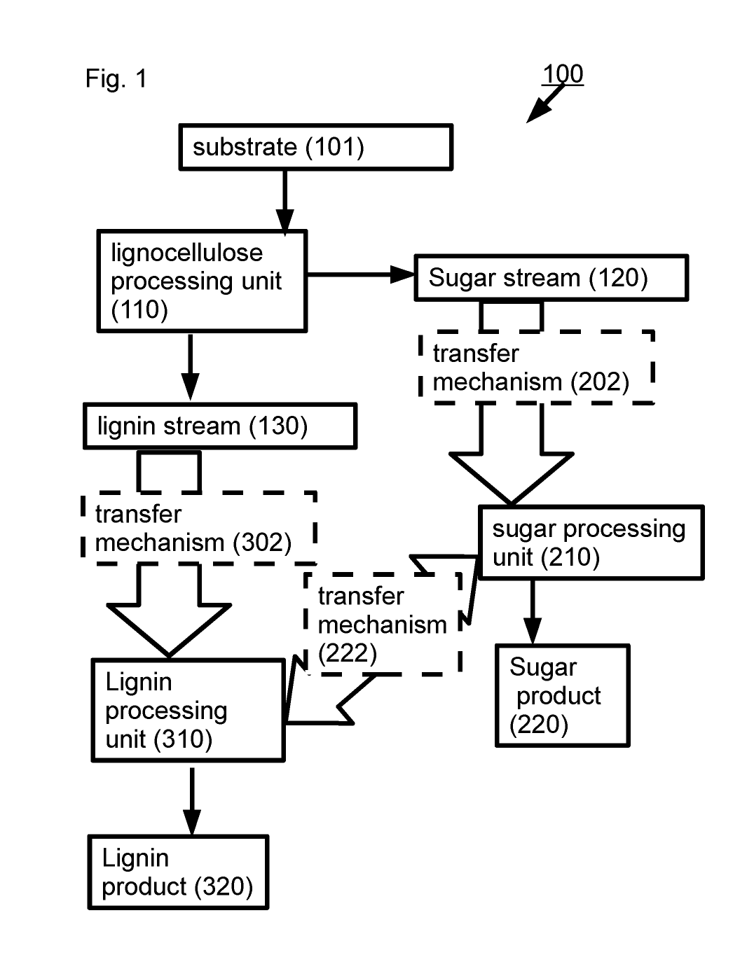

FIG. 1 is a schematic representation of various exemplary embodiments of the invention indicating various types of processing units and some of the possible relationships between them indicated generally as integrated plant 100.

Depicted exemplary integrated plant 100 includes three types of processing units: Lignocellulose processing unit 110, sugar processing unit 210 and lignin processing unit 310. Each of these units includes multiple functional modules. Exemplary functional modules belonging to the various units are described hereinbelow.

Additional exemplary embodiments of the invention include two of these three units. Alternatively or additionally, more than one of any of the three unit types may be provided in an integrated plant in some embodiments.

In the depicted exemplary embodiment, lignocellulose processing unit 110 processes a lignocellulosic substrate 101 to produce one or more sugar streams 120 and one or more lignin streams 130. According to various exemplary embodiments of the invention, the processing includes hydrolysis of cellulose and hemicellulose by various means. In some embodiments, the hydrolysis is with a mineral acid, for example HCl.

Lignocellulosic substrate 101 can include, for example, one or more of: softwood chips, hardwood chips, whole tree chips from softwood or hardwood, grass, agricultural residue, cork, energy crops, cassaya residues, municipal solid waste, industrial solid waste, sludge from paper manufacture, yard waste, forestry waste, field remains and crop processing residues. Field remains include, but are not limited to, corn stover and post harvest plants (e.g. soybean and/or cotton and/or rapeseed). Crop processing residues includes, but is not limited to sugar cane bagasse and empty palm fruit bunches.

Exemplary lignocellulose processing units and/or exemplary modules belonging to such units are described in co-pending applications PCT/US2011/057552; PCT/US2011/046153; PCT/US2011/064237; IL 211093; U.S. 61/524,839; U.S. 61/533,088 and U.S. 61/539,873; each of which is fully incorporated herein by reference.

In the depicted exemplary embodiment, sugar processing unit 210 processes sugar steam 120 (only one is depicted for simplicity, although two or more may actually be produced) to produce one or more sugar products 220. Exemplary sugar processing methods and systems suitable for use in sugar processing unit 210 are described in co-pending application PCT/US2011/050435 which is fully incorporated herein by reference.

In the depicted exemplary embodiment, lignin processing unit 310 processes lignin stream 130 (only one is depicted for simplicity, although two or more may actually be produced) to produce one or more lignin products 320. Exemplary lignin processing methods and modules suitable for use in lignin processing unit 310 are described in co-pending applications PCT/IL2011/000424; U.S. 61/552,402; U.S. 61/552,402; U.S. 61/559,529 and U.S. 61/561,181; each of which is fully incorporated herein by reference.

In the depicted exemplary embodiment, various transfer mechanisms transfer materials and/or energy from one unit to another. These mechanisms are depicted as arrows between units for simplicity, although in actual embodiments the mechanisms often transfer material (or energy) from a specific module in a unit of origin to a specific module in a receiving unit.

As used in this specification and the accompanying claims the term “transfer mechanism” includes, but is not limited to, pipes and/or pumps and/or conveyor belts and/or flow regulators and/or flow splitters and/or tanks and/or carts and/or trucks and/or trains. According to various exemplary embodiments of the invention a transfer mechanism transfers a material and/or energy over a distance of less than 5.01 km, 4 km, 3 km, 2 km, 1 km, 500 m, or even 250 m or intermediate or shorter distances.

In the depicted embodiment, transfer mechanism 202 transfers one or more sugar streams 120 to one or more sugar processing units 210.

In the depicted embodiment, transfer mechanism 302 transfers one or more lignin streams 120 to one or more lignin processing units 310.

In the depicted embodiment, transfer mechanism 222 transfers one or more material between one or more sugar processing units 210 and one or more lignin processing units 310.

Exemplary integration types are explained in greater detail hereinbelow.

Exemplary Integration of Lignocellulose Processing and Sugar Processing

In some embodiments, an integrated plant includes one or more lignocellulose processing units 110 (FIG. 1) producing one or more sugar streams 120 (each sugar stream including one or more sugars) and one or more sugar processing units 210 processing one or more sugars from one or more of sugar streams 120 into one or more sugar product 220 and at least one transfer mechanism 202 transferring one or more of sugar stream(s) 120 to one or more of sugar processing units 210 over a distance of 5 km or less.

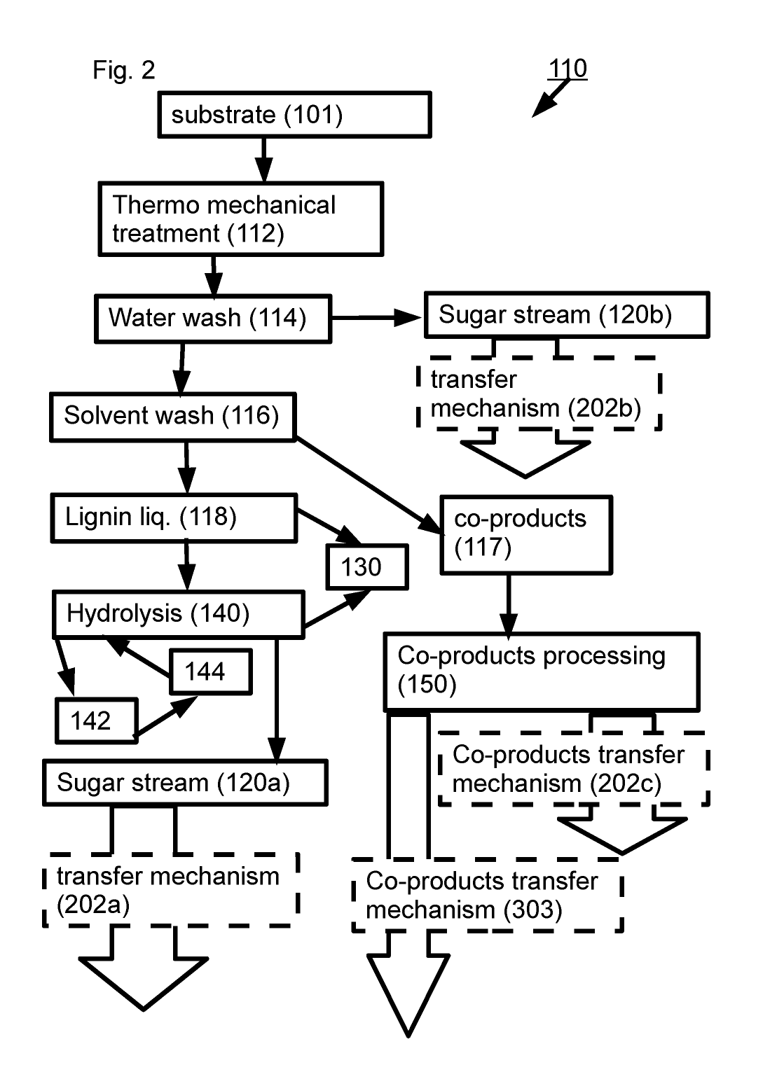

FIG. 2 depicts an exemplary lignocellulose processing unit 110 in greater detail.

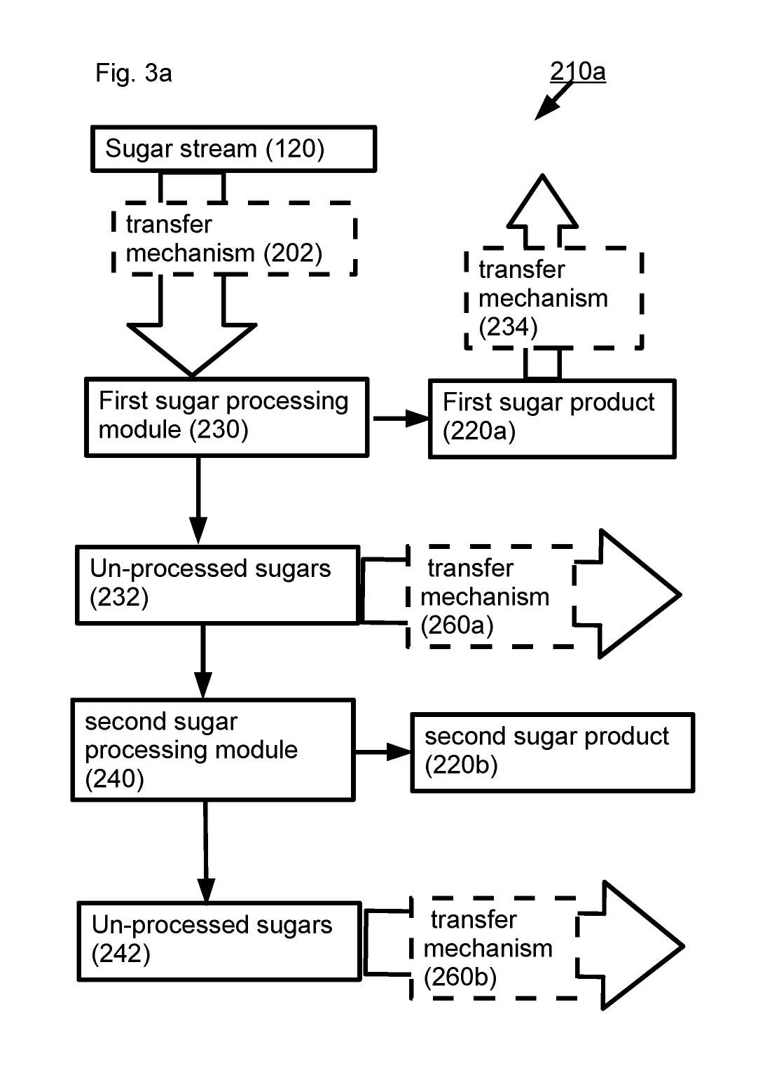

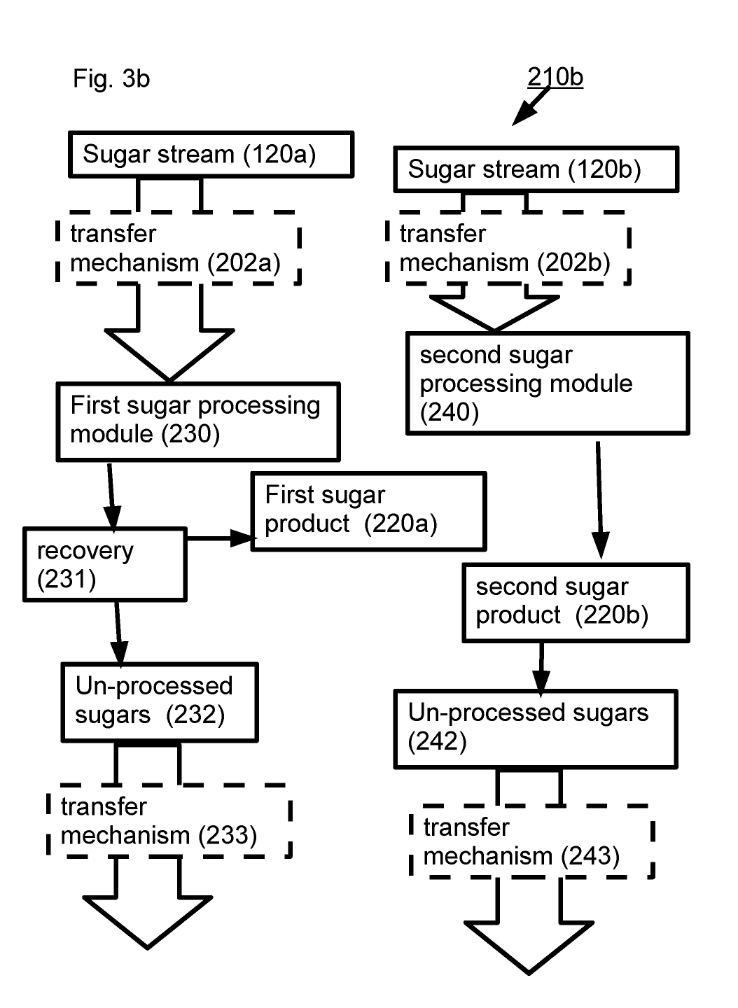

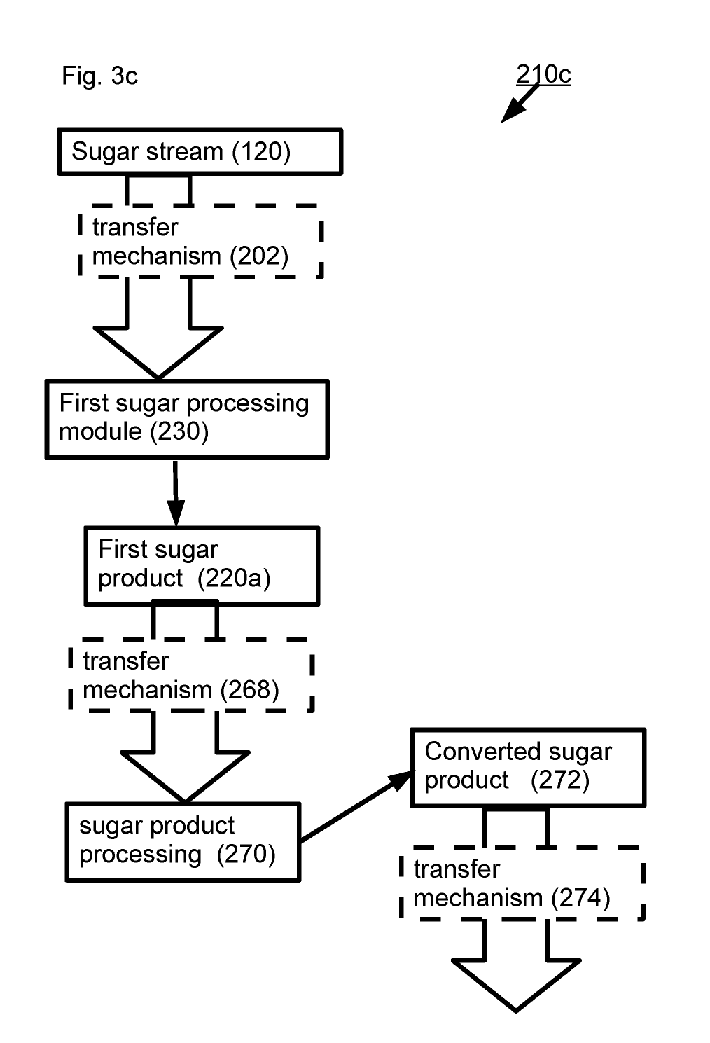

FIGS. 3a, 3b and 3c depict exemplary sugar processing units 210 in greater detail (indicated as 210a; 210b and 210c respectively).

In many embodiments, sugar stream 120 is a hydrolyzate resulting from hydrolysis of substrate 101 in lignocellulose processing unit 110. Optionally, sugar stream 120 includes two or more separate streams. In some embodiments, one or more of streams 120 is enriched for hemicellulose sugars and one or more other streams 120 is enriched for glucose.

According to various exemplary embodiments of the invention lignocellulose processing unit 110 includes one or more hydrolysis reaction modules (140; FIG. 2). According to various exemplary embodiments of the invention hydrolysis reaction modules 140 perform hydrolysis with a mineral acid, hydrolysis with a reactive fluid or enzymatic hydrolysis.

As used in this specification and the accompanying claims the term “reactive fluid” has the meaning ascribed to it in WO 2010/009343; paragraph [0058]:
“ . . . a fluid that is at a temperature higher than the boiling point of the liquid state of the fluid under atmospheric pressure (1 atm). The reactive fluid may be a liquid, a gas, a supercritical fluid, or a mixture of these. For example, water at a temperature above 100° C. and under atmospheric pressure is considered a reactive fluid. Supercritical, near critical, and sub-critical fluids are reactive fluids, illustrative examples including but not limited to sub-critical water, near critical water, supercritical water, supercritical ethanol, and supercritical CO₂.”

WO 2010/009343 is fully incorporated herein by reference.

Exemplary hydrolysis condition are described in PCT/US2011/057552; WO 2010/009343; WO 2011/091044; WO 2010/113130; U.S. Pat. No. 4,349,66; U.S. Pat. No. 4,608,245; U.S. Pat. No. 4,837,315; U.S. Pat. No. 5,188,673; U.S. Pat. No. 5,176,832; U.S. Pat. No. 5,580,389; U.S. Pat. No. 4,384,897; U.S. Pat. No. 4,278,471; U.S. Pat. No. 4,237,110; U.S. Pat. No. 4,608,245; U.S. Pat. No. 4,645,658; U.S. Pat. No. 5,782,982; U.S. Pat. No. 5,580,389; U.S. Pat. No. 5,820,687; U.S. Pat. No. 6,419,828; U.S. Pat. No. 4,237,110; each of which is fully incorporated herein by reference.

In some embodiments, lignocellulose processing unit 110 pre-treats substrate 101 prior to introducing the substrate into hydrolysis module 140.

FIG. 2 is a more detailed schematic representation of lignocellulose processing unit 110 according to some embodiments of the invention which depicts exemplary pre-treatments, and their corresponding modules. According to various embodiments of the invention the various optional pretreatments can be performed serially in any order and/or concurrently.

In the depicted embodiment, a transfer mechanism (not depicted) delivers substrate 101 to a thermo-mechanical treatment module 112 of lignocellulose processing unit 110 which applies a predetermined pressure-temperature-time profile to substrate 101. According to various exemplary embodiments of the invention the predetermined pressure-temperature-time profile includes steam explosion and/or expeller treatment (e.g. using a plug screw feeder) and/or ammonia fiber explosion (AFEX). Optionally, the plug screw feeder treatment includes use of an impressafiner apparatus (Andritz; Graz, Austria). A description of AFEX is provided by Taherzade and Karimi (Int. J. Mol. Sci. (2008) 9: 1621-1651) which is fully incorporated herein by reference.

Exemplary predetermined pressure-temperature-time profiles are described in co-pending applications U.S. 61/552,402 and U.S. 61/558,374; each of which is fully incorporated herein by reference.

In some exemplary embodiments of the invention, application of a predetermined pressure-temperature-time profile produces a disrupted substrate.

In the depicted exemplary embodiment, thermo-mechanical treatment module 112 transfers substrate 101 to a water wash module 114. Water wash module 114 washes substrate 101 with an aqueous solution, optionally containing a weak acid (e.g. sulfurous and/or acetic and/or phosphorous acid). In some embodiments, liquid exits water wash module 114 as a separate sugar stream 120b which is transferred by a transfer mechanism 202b to sugar processing unit 210. In some embodiments, sugar stream 120b is rich in hemicellulose sugars such as xylose, arabinose and mannose.

In the depicted exemplary embodiment, water wash module 114 transfers substrate 101 to a solvent wash module 116. Solvent wash module 116 washes substrate 101 with a water soluble organic solvent, optionally containing a weak acid (e.g. sulfurous and/or acetic and/or phosphorous acid). In some embodiments, the solvent is provided as a re-cycled extractant containing a mixture of solvent and water. In some exemplary embodiments of the invention, the solvent includes an alcohol and/or ketone with less than 5 carbon atoms. Optionally, the solvent includes acetone.

Liquid exiting solvent wash module 116 is a solvent/water miscella including lignocellulose co-products 117. Co-products 117 may include, for example, resin(s) and/or pitch and/or tall oil(s) and/or terpene(s) and/or other volatile organic compound(s) and/or proteinaceous materials and/or ash. The qualitative and/or quantitative composition of co-products 117 may vary depending upon wash conditions in modules 116 and/or 114 and/or the nature of the thermo-mechanical treatment applied in module 112 and/or the initial composition of substrate 101. Solvent wash module 116 transfers the miscella containing co-products 117 to a co-products processing module 150.

According to various exemplary embodiments of the invention co-products processing module 150 separates co-products 117 from solvent and/or water and/or from one another. This separation process is described in greater detail in co-pending application PCT/US2011/064237; which is fully incorporated herein by reference.

Co-products processing module 150 provides one or more effluent streams to one or more transfer mechanisms. In the depicted exemplary embodiment, co-products processing module 150 transfers one or more co-products 117 to sugar processing unit 210 via co-products transfer mechanism 202c. Alternatively or additionally, one or more co-products 117 are anaerobically digested, either in co-products processing module 150 or in a separate module (e.g. in sugar processing unit 210). Alternatively or additionally, in some embodiments, co-products processing module 150 transfers one or more co-products 117 to lignin processing unit 310 via co-products transfer mechanism 303. Exemplary co-products 117 include, but are not limited to, furfurals, tall oils, resins, pitch, ash, methanol and acetic acid.

In some embodiments, lignocellulose co-products 117 includes acetic acid and transfer mechanism 202c functions as an acetic acid transfer mechanism transferring acetic acid to one or more of sugar processing units 210. In some embodiments, unit 210 processes acetic acid to make ethyl acetate.

Alternatively or additionally, lignocellulose co-products 117 include methanol and transfer mechanism 202c functions as a methanol transfer mechanism transferring methanol to one or more of sugar processing units 210. In some embodiments, unit 210 processes methanol with one or more fatty acids (e.g. by fermentation) to make methyl esters. Optionally, the resultant methyl esters are used for biodiesel.

Alternatively or additionally, lignocellulose co-products 117 include one or more tall oils and transfer mechanism 202c functions as a tall oil transfer mechanism transferring tall oils to one or more of sugar processing units 210. In some embodiments, unit 210 processes methanol with one or more fatty acids (e.g. by fermentation) to make methyl esters. Optionally, processing units 210 processes the tall oils with methyl esters (e.g. for biodiesel production).

Optionally, lignocellulose processing unit includes a lignin liquefaction module 118 which dissolves and/or de-polymerizes lignin in substrate 101. In some embodiments, dissolving and/or de-polymerizing lignin includes contacting with an alkaline solution (e.g. ammonia) and/or an organic solvent and module 118 includes components which provide the solution and/or solvent and perform the contacting. Optionally, the solvent includes an aqueous phase reforming (APR) product.

In those embodiments which include module 118, this module produces lignin stream 130 which is transferred by transfer mechanism 302 (FIG. 1) to lignin processing unit 310 (FIG. 1). In some embodiments, lignin liquefaction module 118 employs ammonia to dissolve lignin. Optionally, this ammonia is recovered in lignin processing unit 310 (e.g. by an ammonia distillation module) and recycled by an ammonia transfer mechanism (not depicted) to module 118 for re-use.

In the depicted exemplary embodiment, lignin liquefaction module 118 transfers residual cellulose from substrate 101 to hydrolysis module 140. Hydrolysis module 140 hydrolyzes the cellulose to soluble sugars (typically a mixture of monomeric glucose and oligomers) to produce sugar stream 120a. In some embodiments, sugar stream 120b has not been previously removed, and a single sugar stream 120 containing hemicellulose and cellulose sugars exits hydrolysis module 140. Transfer mechanism 202a transfers the sugar stream exiting hydrolysis module 140 to sugar processing unit 210.

Hydrolysis module 140 is depicted as a single unit producing sugar stream 120a for clarity. However, in many embodiments, hydrolysis module 140 contains many sub-modules.

For example, in embodiments in which hydrolysis module 140 employs HCl for hydrolysis it may include a main hydrolysis reactor as described in PCT/US2011/057552 operating in co-operation with a de-acidification module 142 and solvent recycling module 144 as described in PCT/US2011/046153. Modules 142 and/or 144 recycle HCl to hydrolysis module 140 for re-use in processing additional substrate 101. In some embodiments, this recycling includes extraction of the HCl with a solvent. Optionally, the solvent is treated with lime to remove contaminants. Alternatively or additionally, in some embodiments, this recycling includes distillation. Optionally, distillation equipment in lignocellulose processing unit 110 also receives and processes streams originating in lignin processing unit 310 (FIG. 1). Alternatively or additionally, hydrolysis module 140 optionally incorporates solvent extraction with secondary hydrolysis and/or ion exchange separation as described in IL 211093 and/or U.S. 61/524,839 and/or U.S. 61/533,088 and/or U.S. 61/539,873. Each of the applications mentioned in this paragraph is fully incorporated herein by reference.

According to various exemplary embodiments of the invention sugar processing unit 210 (FIG. 1) performs processing including biological processing (e.g. fermentation) and/or chemical processing (optionally catalyzed reactions) and/or enzymatic reactions. In some exemplary embodiments of the invention, a fermentation product is subject to chemical conversion. One example of chemical processing is hydrogenation of sugars to form corresponding alcohols (e.g. xylose to xylitol and/or glucose to sorbitol and/or a mixture of sugars to “polyol molasses”). According to various exemplary embodiments of the invention multiple processing reactions are conducted. Optionally, these multiple processing reactions are conducted in parallel and/or in series (either within a single sugar processing unit, or in multiple modules of a single sugar processing unit).

For example, in some embodiments, a single sugar processing unit 210 includes two or more fermentation modules arranged in series or two or more fermentation modules arranged in parallel. Alternatively or additionally, in some embodiments, a single sugar processing unit 210 includes a fermentation module followed by chemical processing module which processes unfermented sugars. In some embodiments, hemicellulose sugars are processed separately from cellulose sugars (see sugar streams 120b and 120a of FIG. 2 and accompanying explanation)

FIG. 3a is a schematic representation of an exemplary sugar processing unit indicated generally as 210a. Depicted exemplary module 210a includes sugar processing modules (230 and 240) arranged in series. In the depicted exemplary embodiment, one or more first sugar processing modules 230 processes a first sugar delivered by transfer mechanism 202 as part of sugar stream 120 into a first sugar product 220a. For example, in some embodiments, sugar stream 120 includes glucose and first sugar processing module 230 is a fermentation module containing microorganisms that ferment glucose to produce first sugar product 220a in the form of ethanol. The ethanol can be separated from unprocessed sugars 232, for example by distillation.

According to various exemplary embodiments of the invention unprocessed sugars 232 contain one or more potentially valuable sugars which can be recovered as described below. In those embodiments in which first sugar processing module 230 is a fermentation module, unprocessed sugars 232 may be present as part of a spent culture medium containing non-sugar nutrients and/or cellular material. Optionally, some or all of the non-sugar nutrients and/or cellular material are removed and transferred by transfer mechanism 260a to an additional processing module. For example, a portion of the cellular material can be returned to first sugar processing module 230 for an additional round of fermentation. Alternatively or additionally, non-sugar nutrients and/or cellular material can be routed to a chemically catalyzed processing unit (optionally located in lignin processing unit 310 and/or in sugar processing unit 210, FIG. 1). According to various exemplary embodiments of the invention separation includes crystallization of un-processed sugars and/or centrifugation and/or filtration. Optionally, crystallization of un-processed sugars is facilitated by adding ethanol formed by fermentation in first sugar processing module 230.

Depicted exemplary sugar processing unit 210a also includes one or more second sugar processing modules 240 processing a second sugar into one or more second sugar products 220b. According to various exemplary embodiments of the invention second sugar processing modules 240 perform biological and/or chemical and/or enzymatic processes.

For example, in some embodiments the second sugar is xylose. Optionally, the xylose is crystallized from the unprocessed sugars 232 and separated as described above and second sugar processing module 240 hydrogenates the xylose to form second sugar product 220b in the form of xylitol. In the depicted exemplary embodiment, any remaining unprocessed sugars 242 (optionally with non-sugar material) are removed and transferred by transfer mechanism 260b to an additional processing module. For example, non-sugar material can be routed to a chemically catalyzed processing unit (optionally located in lignin processing unit 310 and/or in sugar processing unit 210, FIG. 1).

FIG. 3b is a schematic representation of an exemplary sugar processing unit indicated generally as 210b. Depicted exemplary module 210b includes sugar processing modules (230 and 240) arranged in parallel to process sugar streams 120a and 120b delivered by transfer mechanisms 202a and 202b respectively.

For example, if sugar streams 120b and 120a originated from wash module 114 and hydrolysis module 140 respectively (FIG. 2), 120b contains a relatively high proportion of hemicellulose sugars such as xylose and 120a is glucose rich.

In some embodiments, the processing is similar to that described above for sugar processing unit 210a (FIG. 3a) except that glucose in sugar stream 120a is fermented to ethanol separately and xylose in sugar stream 120b is crystallized and hydrogenated without prior processing of glucose.

In other exemplary embodiments of the invention, glucose in sugar stream 120a is subject to homolactic fermentation in first sugar processing module 230 to produce lactic acid as a first sugar product 220a. In the depicted exemplary embodiment, recovery module 231 separates first sugar product 220a (e.g. lactic acid) from unprocessed sugars 232 which are then transferred by transfer mechanism 233. According to various exemplary embodiments of the invention transfer mechanism 233 transfers unprocessed sugars 232 to lignin processing unit 310 (FIG. 1) and/or to hydrolysis module 140 (FIG. 2) of lignocellulose processing unit 110. Optionally, oligomeric sugars in unprocessed sugars 232 are hydrolyzed in module 140 to monomeric sugars which return to a sugar processing unit 210 (FIG. 1) as part of a sugar stream 120.

In some embodiments, second sugar processing module 240 crystallizes and hydrogenates mannose in sugar stream 120b to produce second sugar product 220b in the form of mannitol. Unprocessed sugars 242 are transferred by transfer mechanism 243, for example to lignin processing unit 310 (FIG. 1) and/or to a chemical conversion module in sugar processing unit 210.

Referring again to FIG. 3a in some embodiments, sugar processing unit 210a includes at least one transfer mechanism 234 transferring a material from one or more of sugar processing units 210a to one or more of lignocellulose processing unit 110 (FIG. 1). In the depicted exemplary embodiment, transfer mechanism 234 transfers ethanol (first fermentation product 220a). Optionally, the ethanol is used in lignin separation and/or as a solvent in solvent wash module 116 (FIG. 2). In other exemplary embodiments of the invention, first sugar product 220a is an enzyme produced by fermentation in first sugar processing module 230. In some embodiments, such an enzyme is used to hydrolyze hemicellulose and/or cellulose and/or to treat a waste stream.

FIG. 3c is a schematic representation of a sugar processing unit including a sugar product processing module depicted generally as 210c. In some embodiments, sugar processing involves two or more reactions, only some of which use sugar as an input. Optionally, one or more reactions uses a sugar product (e.g. 220) as an input.

In the depicted exemplary embodiment, sugar processing unit 210c includes one or more first sugar processing modules 230 and one or more sugar product processing modules 270.

In the depicted exemplary embodiment, transfer mechanism 202 transfers sugar stream 120 to processing module 230.

In some embodiments, sugar processing modules 230 are fermentation modules and sugar product processing modules 270 are chemical conversion modules.

For example, in some embodiments, sugar processing module 230 ferments glucose to produce lactic acid (first sugar product 220a) and sugar product processing module 270 polymerizes lactic acid into converted sugar product 272 in the form of poly-lactic acid (PLA).

In other embodiments, first sugar processing module 230 produces isobutanol as first sugar product 220a and sugar product processing module 270 dehydrates the isobutanol to butane and polymerizes the butane to fatty acids. Optionally, the fatty acids can be reacted with methanol in module 270 to produce biodiesel as a final converted sugar product 272.

In some embodiments, transfer mechanism 268 transfers a sugar product (e.g. 220a) to one or more sugar product processing modules 270.

In other embodiments of the invention, one first sugar processing module 230 produces acetic acid as first sugar product 220a and another first sugar processing module 230 produces ethanol as first sugar product 220a. According to these embodiments sugar product processing module 270 produces ethyl acetate (e.g. via Fischer esterification) from ethanol and acetic acid as converted sugar product 272.

In other embodiments of the invention, one first sugar processing module 230 produces p-xylene by chemical conversion as first sugar product 220a and another first sugar processing module 230 produces ethanol as first sugar product 220a. According to these embodiments sugar product processing module 270 converts the ethanol to ethylene and reacts it with the p-xylene to produce PET (Polyethylene Terephthalate) as converted sugar product 272.

In some embodiments, transfer mechanism 274 transfers converted sugar product 272 to another module (e.g. a sugar processing module 230 or another sugar product processing module 270 or a module in lignin processing unit 310 (FIG. 1) or a module in lignocellulose processing unit 110 (FIG. 1) for additional processing.

In some embodiments, first sugar processing module 230 is a fermentation module which produces one or more amino acids (e.g. methionine and/or lysine and/or tryptophan and/or valine) as first sugar product 220a. In some of these embodiments, transfer mechanism 268 transfers the amino acid to a feed mill (serving as sugar product processing module 270) where it is combined with other ingredients. According to these embodiments converted sugar product 272 is a food product or animal feed (e.g. crumbled or pelleted livestock feed). Optionally, transfer mechanism 274 loads the feed into sacks, barrels, trucks or railroad cars.

In other embodiments in which first sugar product 220a is an amino acid, sugar product processing module 270 incorporates the amino acid into a culture media which serves as converted sugar product 272. Optionally, transfer mechanism 274 transfers at least a portion of the culture media to one or more fermentors serving as first sugar processing modules 230 and/or second sugar processing modules 240 (e.g. FIGS. 3a and 3b).

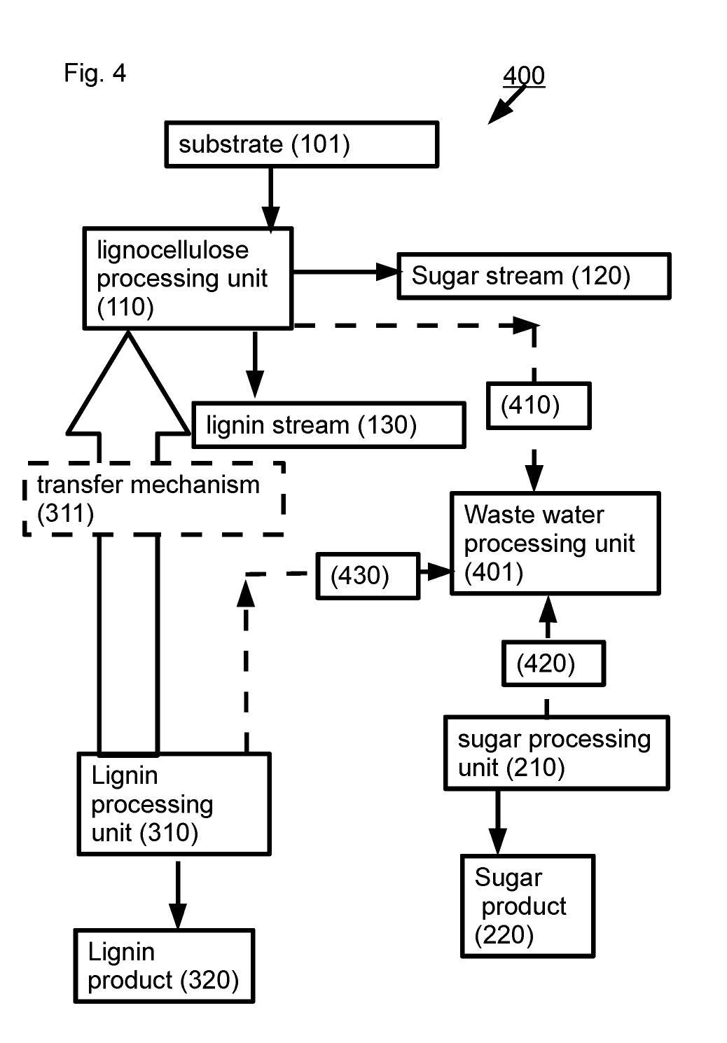

FIG. 4 is schematic representation of additional embodiments of an integrated plant depicted generally as 400. In the depicted exemplary embodiment, many of the transfer mechanisms depicted in FIG. 1 are not shown for clarity, although they may still be present. Integrated plant 400 includes at least two of; one or more lignocellulose processing units 110, one or more sugar processing units 210 and one or more lignin processing units 310 as described hereinabove and/or hereinbelow.

In addition, integrated plant 400 includes a waste water processing unit 401. Optionally, waste water processing unit 401 is integrated into lignocellulose processing unit 110 and/or into sugar processing unit 210 and/or into lignin processing unit 310. In some embodiments, waste water processing unit 401 processes one or more waste streams 410 from one or more of lignocellulose processing units 110. Alternatively or additionally, in some embodiments water processing unit 401 processes one or more waste streams 420 originating from one or more of sugar processing units 210. Alternatively or additionally, in some embodiments, waste water processing unit 401 processes one or more waste streams 430 originating from one or more of lignin processing units 310. According to various exemplary embodiments of the invention additional transfer mechanisms (not depicted) convey the described waste streams between the units.

Referring again to FIG. 2, in some embodiments, co-products processing module 150 includes one or more generators generating heat from combustion of at least one of co-products 117. In other exemplary embodiments of the invention, generators may be present in other modules and/or in other units.

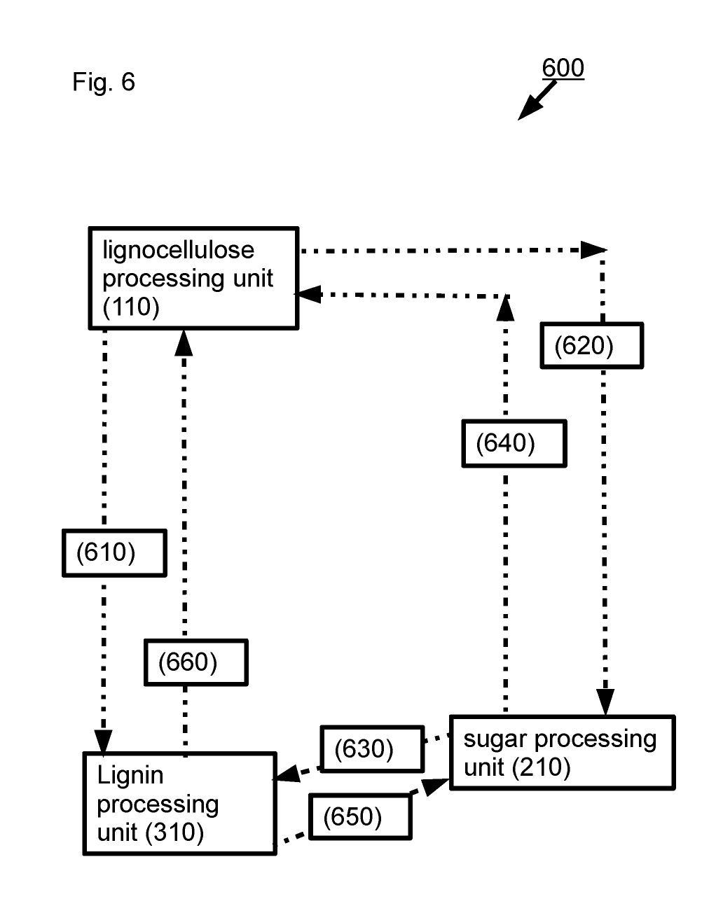

FIG. 6 is a schematic depiction of an integrated plant including two, optionally three, of units 110, 210 and 310 as described hereinabove and/or hereinbelow and indicated generally as 600. FIG. 6 indicates transfer of energy between the various units as dotted/dashed arrows.

In some embodiments, one or more heat transfer mechanisms transfer heat from a generator in one unit to another unit. In the depicted exemplary embodiment, six such heat transfer mechanisms are depicted. According to various exemplary embodiments of the invention an integrated plant contains zero, one, two, three, four, five or all six of the depicted heat transfer mechanisms. For example, in the depicted exemplary embodiment, heat transfer mechanism 620 transfers energy from lignocellulose processing unit 110 to one or more of sugar processing units 210. In some embodiments, heat transfer mechanism 620 includes a pipe conducting steam. Optionally, heat transferred by mechanism 620 is used to control temperature in a fermentor and/or to adjust temperature of a chemical reaction. Alternatively or additionally, an integrated plant includes one or more of the following heat transfer mechanisms:

640 transferring heat from one or more sugar processing units 210 to one or more lignocellulose processing units 110;

630 transferring heat from one or more sugar processing units 210 to one or more lignin processing units 310;

650 transferring heat from one or more lignin processing units 310 to one or more sugar processing units 210;

660 transferring heat from one or more lignin processing units 310 to one or more lignocellulose processing units 110; and

610 transferring heat from one or more lignocellulose processing units 110 to one or more lignin processing units 310.

Referring again to FIG. 2: In some embodiments, a transfer mechanism 202c transfers one or more of co-products 117 to one or more of sugar processing units 210 (e.g. FIG. 1). For example, in some cases co-product 117 includes furfurals and transfer mechanism 202c transfers them to a sugar product processing module 270 (e.g. FIG. 3c) in sugar processing unit 210. Optionally, module 270 is a multistage processing module of the type described in the section entitled “Exemplary multistage chemical processing” hereinbelow.

Referring again to FIG. 3b, in some embodiments, transfer mechanisms 233 and/or 243 transfer one or more sugar processing co-products from one or more of sugar processing units 210 (i.e. processing modules 230 and/or 240) to another unit, e.g. lignocellulose processing unit 110 (e.g. FIG. 1). For example, unprocessed sugars 233 and/or 242 may be transferred to hydrolysis module 140 (FIG. 2).

For example, in some embodiments, sugar processing module 230 ferments glucose to ethanol (first sugar product 220a), which is recovered 231 by distillation. In this example the co-product is spent culture media including unfermented sugars 232. Typically unfermented sugars 232 include soluble oligomers which are potentially available for fermentation after hydrolysis to monomers. In some embodiments, transfer mechanisms 233 and/or 243 transfer unprocessed sugars 232 and/or 242 to hydrolysis module 140 (e.g. FIG. 2) of lignocellulose processing unit 110 for additional hydrolysis.

Alternatively or additionally, in some embodiments, transfer mechanism 233 and/or 243 transfers a sugar product (e.g. 220a or 220b) of sugar processing (e.g. 230 and/or 240) to one or more lignocellulose processing units. For example, in some embodiments, transfer mechanism 233 transfers ethanol (sugar product 220a) to solvent wash module 116 (FIG. 2) to serve as a solvent in extraction of co-products 117.

Exemplary Integration of Lignocellulose Processing and Lignin Processing

Referring again to FIG. 1, in some embodiments the integrated plant includes one or more lignocellulose processing units 110 producing at least one lignin stream 130 and one or more lignin-processing units 310 processing lignin from the at least one lignin stream 130 into a lignin product 320 and at least one transfer mechanism 302 transferring said lignin stream from one or more of lignocellulose processing units 110 to one or more of lignin processing unit 310 over a distance of 5 km or less.

According to various exemplary embodiments of the invention lignin product 320 includes one or more of clean de-acidified lignin (optionally pelletized), carbon fibers, lignosulfonates bio-oil, carboxylic and fatty acids, dicarboxylic acids, hydroxyl-carboxylic, hydroxyl di-carboxylic acids and hydroxyl-fatty acids, methylglyoxal, mono-, di- or poly-alcohols, alkanes, alkenes, aromatics, aldehydes, ketones, esters, phenols, toluenes, xylenes (e.g. para-xylene), fuels, fuel ingredients, liquefied lignin and a composition including a liquid comprising at least 20% lignin by weight.

Alternatively or additionally, lignin product 320 belongs to one or more of the following categories: dispersants, emulsifiers, complexants, flocculants, agglomerants, pelletizing additives, resins, carbon fibers, active carbon, antioxidants, liquid fuel, aromatic chemicals, vanillin, adhesives, binders, absorbents, toxin binders, foams, coatings, films, rubbers and elastomers, sequestrants, fuels, and expanders.

According to various exemplary embodiments of the lignin stream 130 is a liquid stream or a slurry, optionally including acid.

In some embodiments, one or more of lignocellulose processing units 110 produces at least one co-product 117 (FIG. 2) selected from the group consisting of tall oils, ash, resins, pitch and furfurals as described hereinabove.

In some embodiments, the plant includes a transfer mechanism 311 (FIG. 4) transferring a material from one or more of lignin processing units 310 to one or more of lignocellulose processing units 110.

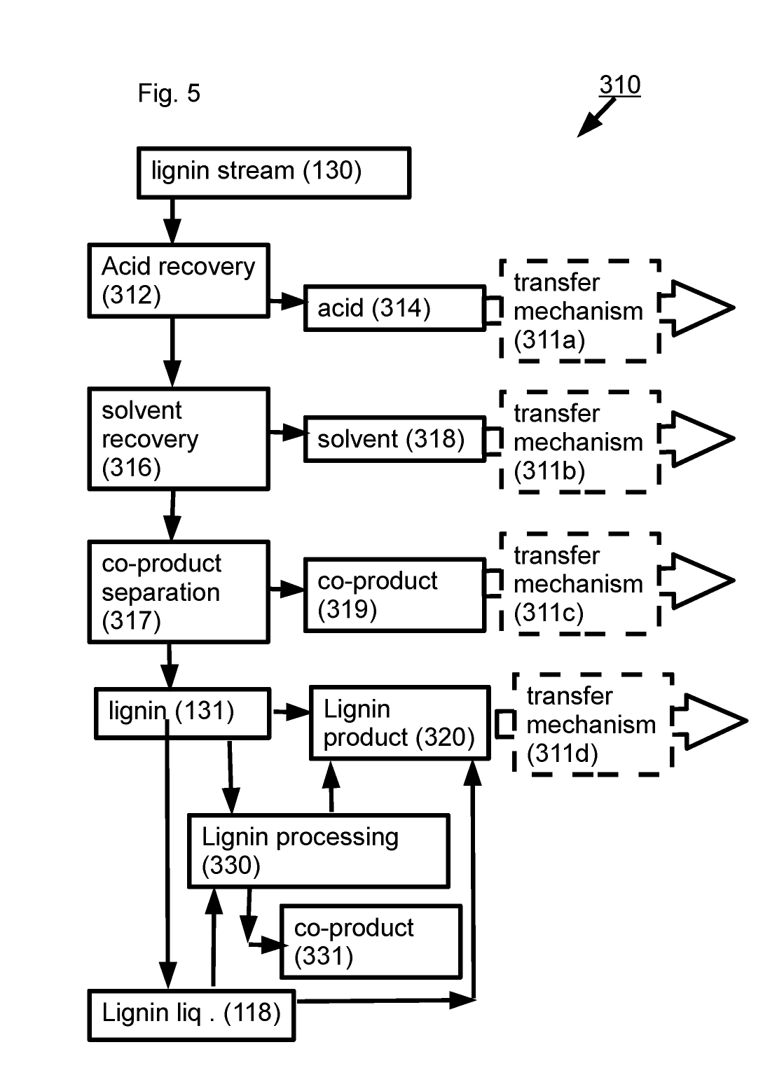

FIG. 5 is a schematic representation of an exemplary lignin processing unit 310 depicting exemplary modules in an exemplary order. According to various exemplary embodiments of the invention the actual modules and/or their order may vary.

Exemplary lignin processing methods are described in co-pending applications U.S. 61/552,402 and U.S. 61/559,529; which are each fully incorporated herein by reference.

In the depicted exemplary embodiment, lignin stream 130 is processed by an acid recovery module 312 and a solvent recovery module 316. These modules are depicted separately for simplicity but may be interconnected in actual practice. Optionally, stream 130 is then processed in a co-product separation module 317. According to various exemplary embodiments of the invention module 317 removes residual sugars and/or residual cellulose as co-products 319 to produce cleaned lignin 131. Optionally, co-products 319 are transferred by transfer mechanism 311c to sugar processing unit 210 (e.g. FIG. 1) and/or lignocellulose processing unit 110 (e.g. FIG. 1). In some embodiments, cleaned lignin 131 serves as lignin product 320. In other exemplary embodiments of the invention, cleaned lignin is processed in lignin processing module 330 to produce lignin product 320 (e.g. pelleted lignin or carbon fibers). Alternatively or additionally, lignin 131 is liquefied by lignin liquefaction module 118.

According to various exemplary embodiments of the invention lignin processing module 330 performs one or more reactions including, but not limited to, hydrolysis, oxidation, reduction, sulfonation, alkylation and mechanical treatment on lignin 131.

In some embodiments, dissolving and/or depolymerizing lignin includes contacting with an alkaline solution (e.g. ammonia) and/or an organic solvent and module 118 includes components which provide the solution and/or solvent and perform the contacting. Optionally, the solvent includes an APR product.

Alternatively or additionally, in some embodiments, depolymerizing lignin includes pyrolysis and/or gasification and/or hydrogenolysis and/or contacting with a supercritical fluid.

In some embodiments, lignin product 320 is transferred by transfer mechanism 311d. This transfer can be, for example, to a multistage chemical processing module (e.g. sugar processing module 230 (FIG. 3a and/or 3b and/or 3c) or sugar product processing module 270 (FIG. 3c)) as described hereinbelow in the section entitled “Exemplary multistage chemical processing”. Optionally, transfer mechanism 311d and/or lignin processing module 330 include size fractionation components which allow control of an average molecular weight and/or degree of polydispersity of lignin product 320 being transferred by mechanism 311d.

In some embodiments, transfer mechanism 311 includes an acid transfer mechanism 311a transferring acid 314 from an acid recovery module 312 of lignin processing unit 310 to one or more of lignocellulose processing units 110 (e.g. FIG. 2). Optionally, Acid recovery module 312 functions also to recover sugars from lignin stream 130. In some embodiments, transfer mechanism 311a transfers sugars to lignocellulose processing unit 110 (e.g. to hydrolysis module 140, FIG. 2) together with acid 314.

Alternatively or additionally, in some embodiments, transfer mechanism 311 includes a solvent transfer mechanism 311b transferring solvent 318 from a solvent recovery module 316 of lignin processing unit 310 to one or more of lignocellulose processing units 110 (e.g. FIG. 2). Optionally, transfer mechanism 311b includes solvent purification components as described in PCT/US2011/46153; which is fully incorporated herein by reference. In some embodiments, solvent purification includes treatment with lime.

Alternatively or additionally, in some embodiments, the plant includes one or more acid-recycle modules (not depicted) adapted to recover acid from one or more lignin processing units 310 and return the acid to one or more of lignocellulose processing units 110 (e.g. FIG. 2).

Systems and methods for acid recovery and/or re-cycling and/or solvent recovery/recycling are described in co-pending applications PCT/IL2011/000424; PCT/US2011/57552; PCT US2011/46153; U.S. 61/539,861; U.S. 61/533,088; U.S. 61/524,839 and Il 211093; each of which is fully incorporated herein by reference.

Referring now to FIG. 4, in some embodiments, the plant includes a waste water treatment unit 401 treating one or more waste streams 410 from one or more lignocellulose processing units 110 and/or one or more waste streams 430 one or more lignin processing units 310.

Referring again to FIGS. 2 and 6, in some embodiments the plant includes a generator (depicted as co-products processing module 150) generating heat from combustion of at least one of co-product 117. Optionally, the heat is provided as steam. This steam may be used, for example, for acid recovery (e.g. in hydrolysis module 140 (FIG. 2), de-solventization of wood (e.g. in module 116, FIG. 2) and/or steam explosion (e.g. in module 112, FIG. 2) and/or in lignin processing unit 310. In some embodiments, the plant includes a heat transfer mechanism 610 transferring heat from the generator to one or more lignin processing units 310. Optionally, mechanism 610 includes a pipe or other conduit transferring steam.

Alternatively or additionally, in some embodiments, lignin processing unit 310 includes a generator generating heat from combustion. In some embodiments, the generator is part of lignin processing module 330 (e.g. FIG. 5). Optionally, module 330 performs pyrolysis and/or gasification of lignin 131. Optionally, the heat is provided as steam. This steam may be used, for example, for acid recovery and/or solvent purification (modules 142 and 144 respectively (FIG. 2)) and/or steam explosion (e.g. in module 112 (FIG. 2)). In some embodiments, the plant includes a heat transfer mechanism 660 transferring heat from the generator to one or more lignocellulose processing units 110 (FIG. 2). Optionally, mechanism 660 includes a pipe or other conduit transferring steam.

Referring now to FIG. 2, alternatively or additionally, in some embodiments the plant includes a transfer mechanism 303 transferring one or more of co-products 117 from one or more of lignocellulose processing units 110 to one or more of lignin processing units 310 (FIG. 5).

Referring now to FIG. 4, alternatively or additionally, in some embodiments the plant includes one or more transfer mechanisms transferring a waste stream 430 from one or more of lignin processing units 310 to one or more of waste-water treatment units 401.

Referring now to FIG. 5, in some embodiments, the plant includes one or more transfer mechanisms 311c transferring a lignin processing co-product (e.g. 319 and/or 331) from one or more of lignin processing units 310 to one or more of lignocellulose processing units 110 (FIG. 2). For example, co-product 331 may include an alkene. Optionally, the alkene is burned to generate energy in lignocellulose processing unit 110. Alternatively or additionally, co-product 331 (or 319) may include organic contaminants to be treated in a wastewater processing unit 401 (FIG. 4) associated with lignocellulose processing unit 110. Alternatively or additionally, in some embodiments the plant includes one or more transfer mechanisms 311d transferring at least a portion of lignin product 320 from one or more of lignin processing units 310 to one or more of lignocellulose processing units 110 (FIG. 2). For example, solvents produced by APR may be transferred to a lignin liquefaction module 118 (FIG. 2) in lignocellulose processing unit 110 (FIG. 2).

Exemplary Tripartite Processing Integration

Referring again to FIG. 1, in some embodiments the integrated plant includes one or more lignocellulose processing units 110 producing one or more sugar streams 120 and one or more lignin streams 130 and one or more lignin-processing units 310 processing one or more of lignin streams 310 into a lignin product 320 and one or more sugar processing units 210 processing one or more of sugar streams 120 into a sugar product 220 and at least one transfer mechanism 202 transferring one or more of sugar stream(s) 120 the sugar processing units 210 over a distance of 5 km or less and at least one transfer mechanism 302 transferring lignin stream 130 from the lignocellulose processing units 110 to the lignin processing units 310 over a distance of 5 km or less.

According to various embodiments of the invention each sugar processing unit 210 can include chemical processing (e.g. multistage chemical processing) and/or biological processing (e.g. fermentation) and/or enzymatic processing.

As described hereinabove, in some embodiments, lignocellulose processing units 110 optionally produce at least one co-product 117 (FIG. 2).

In some embodiments, sugar processing units 210 include one or more second sugar processing units 210 processing one or more second sugars into one or more second sugar products.

Referring again to FIG. 2, in some embodiments, a single lignocellulose processing unit 110 is integrated with two separate sugar processing units 210 (FIG. 1). For example, a single lignocellulose processing unit 110 can include a transfer mechanism 202b transferring sugar stream 120b to one sugar processing unit 210 and a transfer mechanism 202a transferring sugar stream 120a to a different sugar processing unit 210.

Referring again to FIG. 6, in some embodiments, the integrated plant includes at least one energy transfer mechanism transferring energy between the various units.

In some embodiments, energy transfer mechanism 610 transfers energy from at least one lignocellulose processing unit 110 to at least one lignin processing 310. Alternatively or additionally, in some embodiments, energy transfer mechanism 620 transfers energy from at least one lignocellulose processing unit 110 to at least one sugar processing unit 210. Alternatively or additionally, in some embodiments, energy transfer mechanism 630 transfers energy from at least one sugar processing unit 210 to at least one lignin processing unit 310. Alternatively or additionally, in some embodiments, energy transfer mechanism 640 transfers energy from at least one sugar processing unit 210 to at least one lignocellulose processing unit 110. Alternatively or additionally, in some embodiments, energy transfer mechanism 650 transfers energy from at least one lignin processing unit 310 to at least one sugar processing unit 210. Alternatively or additionally, in some embodiments, energy transfer mechanism 660 transfers energy from at least one lignin processing unit 310 to at least one lignocellulose processing unit 110. In some embodiments, an energy transfer mechanism transfers energy as heat (e.g. as steam flowing through a pipe). Alternatively or additionally, in some embodiments, an energy transfer mechanism transfers energy as electricity (e.g. by using steam to drive a turbine which generates an electric current). Alternatively or additionally, in some embodiments, an energy transfer mechanism transfers energy as mechanical energy (e.g. by using a stream to turn a wheel connected to a drive train).

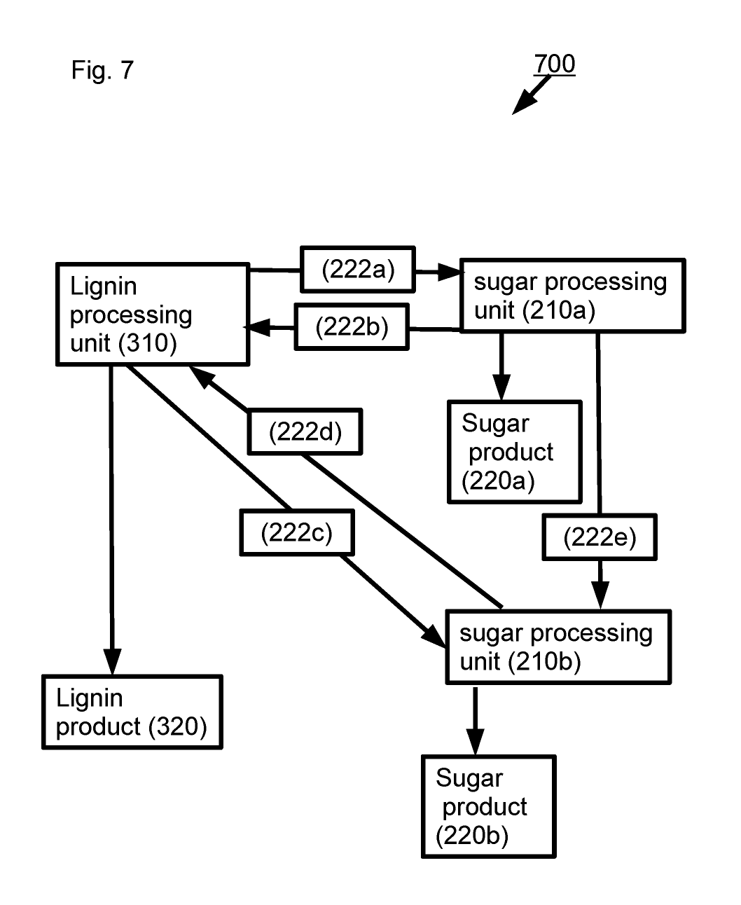

FIG. 7 is a schematic diagram of an integrated plant including at least one lignin processing unit 310 and at least two sugar processing units 210a and 210b indicated generally as 700.

In the depicted exemplary embodiment, plant 700 includes transfer mechanism 222a transferring a material from one or more of lignin processing units 310 to one or sugar processing units 210a. Alternatively or additionally, in some embodiments, the plant includes one or more transfer mechanisms 222d transferring materials from one or more of lignin processing units 310 one or more different sugar processing units 210b. Alternatively or additionally, in some embodiments, the plant includes a transfer mechanism 311 transferring one or more materials from a lignin processing unit 310 to one or more lignocellulose processing units 110 as described in the context of FIGS. 4 and 5.

Alternatively or additionally, in some embodiments, the plant includes two or more transfer mechanisms 222b and/or 222c transferring materials from two or more separate sugar processing units to one or more lignin processing units 310.

In some embodiments, one or more sugar processing unit 210 is adapted to separate a sugar processing co-product (e.g. un-processed sugars 232 and/or spent culture media) from a sugar product (e.g. 220a) as described hereinabove in the context of FIGS. 3a and 3b. Alternatively or additionally, in some embodiments, sugar processing units 210a and/or 210b include transfer mechanisms of the type described as 233 and/or 243 which transfer a sugar product to one or more lignocellulose processing units.

In some embodiments, one or more of sugar processing units 210 includes one or more chemical sugar conversion modules (e.g. sugar processing module 230 (FIG. 3a and/or 3b and/or 3c)) adapted to chemically convert a sugar to a fermentable intermediate prior to fermenting to produce a sugar product. For example, in some embodiments, sugar processing module 230 converts glucose to maltose. According to these embodiments, an additional sugar processing module (e.g. 240 in FIG. 3a) ferments maltose to a fermentation product (e.g. lactic acid and/or ethanol).

In some embodiments, the integrated plant includes one or more conversion modules (e.g. sugar product processing module 270, FIG. 3c) adapted to convert at least one sugar product (e.g. 220a) produced by fermentation into a converted product 272 (FIG. 3c). Optionally, this conversion can occur in the reaction medium or after separation of sugar product 220a. For example, if sugar product 220a is ethanol, product processing module 270 (FIG. 3c) can convert the ethanol to ethyl halides and/or to ethyl esters and/or to diethyl ether and/or to acetic acid and/or to ethyl amines and/or to butadiene. Alternatively if sugar product 220a is lactic acid product processing module 270 (FIG. 3c) can convert the lactic acid to polylactic acid and/or related products.

Referring again to FIG. 3b, in some embodiments at least one of sugar processing units 210 includes a recovery module 231 adapted to recover unprocessed sugars 232. For example, unprocessed sugars may include sugars which were not fermented in processing module 230. Optionally, sugars 232 are recovered as part of a fermentation broth. Optionally, first sugar product 220 is distilled out of the broth as part of recovery 231. Alternatively or additionally, unprocessed sugars 232 can be separated, e.g. by crystallization. Optionally unprocessed sugars 232 are subject to additional processing. (e.g. sugars 232 may include xylose which can be crystallized and hydrogenated to make xylitol.)

Exemplary Integration of Sugar Processing and Lignin Processing

Referring again to FIG. 1, in some embodiments, the integrated plant includes one or more lignin-processing units 310 processing one or more lignin streams 130; and one or more sugar processing units 210 processing one or more sugar streams 120 into one or more sugar products 220 and at least one transfer mechanism 222 transferring one or more materials between one or more of the sugar processing units 210 and one or more of the lignin processing units 310 over a distance of 5 km or less in either direction. According to various exemplary embodiments of the invention the sugar processing includes chemical processing (e.g. enzymatic) and/or biological processing (e.g. fermentation).

Referring now to FIG. 7, in some embodiments, the plant includes one or more additional sugar processing units (210a and 210b) processing sugar into one or more additional sugar products (220a and 220b). In some embodiments, the plant includes at least one transfer mechanism (222c and/or 222d) transferring one or more materials between one or more of additional sugar processing units (210b) and one or more lignin processing units 310 over a distance of 5 km or less in either direction. Alternatively or additionally, energy transfer mechanisms transfer energy between lignin processing units 310 and sugar processing units 210a and/or 210b in either direction in a manner similar to that described hereinabove in the context of FIG. 6 (630 and/or 650).

In some embodiments, the plant includes a transfer mechanism 222e transferring a material from one or more of sugar processing units 210a to one or more of additional sugar processing units 210b.

Alternatively or additionally, in some embodiments, transfer mechanism 222a includes a hydrogen transfer mechanism transferring hydrogen from one or more of lignin processing units 310 to one or more of sugar processing units 210a. According to these embodiments, hydrogen is a lignin product 320 produced by pyrolysis and/or gasification of lignin in lignin processing module 330 (FIG. 5). Optionally, the hydrogen is used in hydrogenolysis and/or hydrogenation of sugars. In some embodiments, this hydrogenolysis and/or hydrogenation of sugars is followed by APR as explained hereinbelow in the section entitled “Exemplary multistage chemical processing”.

Alternatively or additionally, in some embodiments, transfer mechanism 311d (FIG. 5) transfers liquefied lignin as part of lignin product 320 (FIG. 5) to sugar processing unit 210a and/or 210b. According to various exemplary embodiments of the invention, the liquefied lignin is to hydrogenolysed and/or hydrogenated with sugars and/or is subject to APR and/or is subject to one or more conversion steps together with products of an APR reaction performed on sugars.

Referring again to FIG. 7, in some embodiments, the integrated plant includes one or more transfer mechanisms (e.g. 222b and 222c) transferring a material from one or more of sugar processing units 210a and/or 210b to one or more of lignin processing units 310. Optionally, the material being transferred includes a solvent produced by an Aqueous phase reforming (APR) reaction in a sugar product processing module 270 (FIG. 3c) in one or more of sugar processing units 210a and/or 210b. In some embodiments, lignin processing module 330 (FIG. 5) employs this solvent as a reagent in processing lignin 131 (FIG. 5).

Alternatively or additionally, in some embodiments, transfer mechanisms 222b and/or 222c transfer an alcohol. According to various exemplary embodiments of the invention the alcohol is formed by fermentation (e.g. in a sugar processing module 230; FIG. 3c) and/or by APR (e.g. in a sugar product processing module 270; FIG. 3c). In some exemplary embodiments acid recovery module 312 (FIG. 5) uses the alcohol to de-acidify lignin. Alternatively or additionally, the alcohol can be recovered after use, e.g. by distillation.

Alternatively or additionally, in some embodiments the plant includes a transfer mechanism 222c transferring a material from one or more of lignin processing units 310 to one or more of additional sugar processing unit 210b. Optionally, the material includes hydrogen. In some embodiments, the hydrogen is used in processing of an APR product to produce a fuel ingredient. Alternatively or additionally, in some embodiments, transfer mechanism 222c transfers liquefied lignin. Optionally, the liquefied lignin is added to APR products and proceeds to catalytic reactions in sugar product processing 270 (FIG. 3c) with products 220 of sugar processing modules 230 (FIGS. 3a and/or 3b and/or 3c).

Alternatively or additionally, in some embodiments, the plant includes a transfer mechanism (e.g. 222b and/or 222c) transferring a material from at least one sugar processing units 210a and/or 210 b into one or more of lignin processing units 310. In some embodiments, the material includes polyols (converted sugar product 272; FIG. 3c) produced by sugar product processing (e.g. by hydrogenation). In some embodiments, lignin liquefaction module 118 (FIG. 5) uses the polyols to dissolve lignin. Alternatively or additionally, polyols are transferred by a transfer mechanism to lignin liquefaction module 118 (FIG. 2) in lignocellulose processing unit 110 (FIG. 2) which uses them to dissolve lignin prior to hydrolysis 140 (FIG. 2) of cellulose.

Referring now to FIG. 3a, in some embodiments, transfer mechanism 260a and/or 260b includes cell separation equipment (e.g. centrifuges and/or filters) which separates cells and/or spent culture media from unprocessed sugars 232 or 242. Optionally, transfer mechanism 260a and/or 260b transfers these cells and/or spent culture media to lignin processing module 330 of lignin processing unit 310 (FIG. 5). Optionally, lignin processing module 330 is a multistage chemical processing module as described hereinbelow.

Exemplary Capacity Statistics

Referring now to FIG. 1, in some embodiments, the one or more lignocellulose processing units 110 process at least 10 tons of lignocellulose per hour.

Alternatively or additionally, in some embodiments, an acid hydrolysis module 140 (FIG. 2) has a capacity of at least 1, 2, 3, 5 or even 10 or more tons of substrate/hour.

Alternatively or additionally, in some embodiments, the one or more transfer mechanisms 202 transfer at least 30,000 tons of sugar/yr from the one or more lignocellulose processing units 110 to the one or more sugar processing units 210.

Alternatively or additionally, in some embodiments, transfer mechanism 302 transfers at least 10,000 tons of lignin/yr from lignocellulose processing unit(s) 110 to lignin-processing unit(s) 320.

Alternatively or additionally, in some embodiments, a distillation capacity of acid recovery module 142 (FIG. 2) and/or solvent purification module 144 (FIG. 2) is at least 1, at least 10, or even at least 20 tons/hour. Alternatively or additionally, the distillation capacity of one or more of these modules is at least 1, at least 10, or even at least 100 cubic meters of liquid to be distilled per hour.

Alternatively or additionally, the one or more sugar processing units 210 have a processing capacity of at least 3 tons of sugar per hour.

Exemplary Multistage Chemical Processing

In some embodiments, a single processing module depicts a multistage process. One example of such a multistage process is a multistage chemical conversion process of the type described in a white paper entitled “Production of Conventional Liquid Fuels from Sugars” by Blommel and Cortwright (2008) which is fully incorporated herein by reference. Briefly the white paper describes a three stage process including hydrogenation or hydrogenolysis; Aqueous-Phase Reforming and one or more additional conversion steps to produce a final product.

“Aqueous-Phase Reforming” or “APR” indicates a catalytic reforming process that generates hydrogen-rich fuels from oxygenated compounds derived from biomass (e.g. glycerol, sugars, sugar alcohols, etc.). Various APR methods and techniques are described in U.S. Pat. No. 6,699,457; U.S. Pat. No. 6,953,873; U.S. Pat. No. 6,964,757; U.S. Pat. No. 6,964,758; U.S. Pat. No. 7,618,612 and PCT/US2006/048030; each of which is fully incorporated herein by reference. As used in this specification and the accompanying claims the terms “aqueous phase reforming” and “APR” generically denote the overall reaction of an oxygenated compound and water to yield a hydrogen stream, regardless of whether the reactions takes place in the gaseous phase or in the condensed liquid phase. “APR hydrogen” shall indicate hydrogen produced by the APR process. APR converts input oxygenated compounds to products including, but not limited to alcohols, ketones, aldehydes, alkanes, organic acids and furans.

Lignin decomposition products (LDPs) can be produced, for example, by pyrolysis and/or hydrogenolysis and/or oxidation and/or contact with a super-critical (or near super-critical) fluid such as water or another solvent. Exemplary methods for production of LDPs are reviewed by Pandey and Kim in “Lignin Depolymerization and Conversion: A Review of Thermochemical Methods” (Chem. Eng. Technol. (2011) 34 (1): 29-41) which is fully incorporated herein by reference. As used in this specification and the accompanying claims the term “LDP” includes, but is not limited to phenols (e.g. phenol, catechol, guaiacol, syringol and cresol), aldehydes (e.g. vanillin and syringaldehyde) and aliphatics (e.g. methane, ethane and branched alkanes).

According to various exemplary embodiments of the invention a sugar processing module (e.g. 230 and/or 240) (FIGS. 3a, 3b, 3c) and/or a sugar product processing module (e.g. 270)(FIG. 3c) and/or lignin processing module 330 (FIG. 5) includes such multistage processing.

Exemplary Fermentation Considerations

According to various exemplary embodiments of the invention sugar processing modules (e.g. 230 and/or 240; FIGS. 3a and/or 3b and/or 3c) ferment one or more sugars in an input sugar stream. In those embodiments where the sugar processing module includes a fermentor, one or more additional materials may be added to sugar stream 120. Exemplary additional materials include, but are not limited to, additional growth medium components (e.g. nitrogen source and/or addition carbon source and/or vitamins and/or minerals) and a biological inoculum. Exemplary biological inoculums include, but are not limited to, yeast, bacteria, fungi and eukaryotic cells (e.g. mammalian and/or plant and/or insect cells). The inoculum optionally includes wild type and/or genetically modified organisms (GMO).

For example, in various embodiments, a sugar stream 120 is used in one or more processes as described in U.S. Pat. No. 7,629,010; U.S. Pat. No. 6,833,149; U.S. Pat. No. 6,610,867; U.S. Pat. No. 6,452,051; U.S. Pat. No. 6,229,046; U.S. Pat. No. 6,207,209; U.S. Pat. No. 5,959,128; U.S. Pat. No. 5,859,270; U.S. Pat. No. 5,847,238; U.S. Pat. No. 5,602,286; and U.S. Pat. No. 5,357,035; the contents of each of which are fully incorporated herein by reference. In various embodiments, the processes described in the above US patents are combined with one or more steps as described herein, for example, with the step of recycling hydrochloric acid.

In some embodiments, the fermentation employs a GMO. GMOs may include, but are not limited to, members of the genera Clostridium, Escherichia, Salmonella, Zymomonas, Rhodococcus, Pseudomonas, Bacillus, Enterococcus, Alcaligenes, Lactobacillus, Klebsiella, Paenibacillus, Corynebacterium, Brevibacterium, Pichia, Candida, Hansenula and Saccharomyces. Hosts that may be particularly of interest include: Oligotropha carboxidovorans, Escherichia coli, Bacillus lichenifonnis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae. Also, any of the known strains of these species may be utilized as a starting microorganism. In various embodiments, the microorganism is an actinomycete selected from Streptomyces coelicolor, Streptomyces lividans, Streptomyces hygroscopicus, or Saccharopolyspora erytlzraea. In various embodiments, the microorganism is an eubacterium selected from Escherichia coli, Pseudomonas flucrescens, Pseudomonas putida, Pseudomonas aeruginosa, Bacillus subtilis, or Bacillus cereus.

In some embodiments, the GMO is a gram-negative bacterium. In some embodiments, the GMO is selected from the genera Zymomonas, Escherichia, Alcaligenes, and Klebsiella. In some embodiments, the recombinant microorganism is selected from the species Escherichia coli, Cupriavidus necator, and Oligotropha carboxidovorans. In some embodiments, the recombinant microorganism is an E. coli strain.

According to various exemplary embodiments of the invention fermentation in a sugar processing module (e.g. 230 and/or 240; FIGS. 3a and/or 3b and/or 3c) produces one or more sugar products (e.g. 220a and/or 220b). Exemplary sugar products which can result from fermentation include, but are not limited to, ethanol, acetic acid, acrylic acid, lactic acid, 3-HP, butanol, amino acids, fatty acids, and fatty alcohols.

It is expected that during the life of this patent many acid recovery and/or solvent purification techniques will be developed and the scope of the invention is intended to include all such new technologies a priori.

As used herein the term “about” refers to ±10%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Specifically, a variety of numerical indicators have been utilized. It should be understood that these numerical indicators could vary even further based upon a variety of engineering principles, materials, intended use and designs incorporated into the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe units in a tripartite plant (110+210+310) can be used to characterize units in a plant with two unit types (110+210; 110+310 and 210+310) and features used to describe units in a plant with two unit types (110+210; 110+310 and 210+310) can be used to characterize units in a tri-partite plant (110+210+310).

It should be further understood that the individual features described hereinabove can be combined in all possible combinations and sub-combinations to produce additional embodiments of the invention. The examples given above are purely illustrative and do not limit the scope of the invention which is defined solely by the following claims. Specifically, the invention has been described in the context of an integrated plant processing a lignocellulosic substrate as well as sugars and/or lignin but also includes integrated plants with units processing additional input materials.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

1. An integrated plant comprising:
(a) one or more lignocellulose processing units producing one or more sugar streams and one or more lignin streams;
(b) one or more lignin-processing units processing one or more of said lignin streams into a lignin product; and
(c) one or more sugar processing units processing one or more of said sugar streams into a sugar product;
(d) at least one transfer mechanism transferring one or more of said sugar stream(s) to one or more of said sugar processing units over a distance of 5 km or less; and
(e) at least one transfer mechanism transferring said lignin stream from one or more of said lignocellulose processing units to one or more of said lignin processing units over a distance of 5 km or less.

2. A plant according to claim 1, wherein said lignocellulose processing units produce one or more lignocellulose co-products.

3. A plant according to claim 1 or claim 2, comprising one or more second sugar processing units processing one or more second sugars into one or more second sugar products.

4. A plant according to any one of claims 1 to 3, comprising at least one energy transfer mechanism transferring energy between units in at least one relationship selected from the group consisting of:
(i) from at least one lignocellulose processing unit to at least one lignin processing unit;
(ii) from at least one lignocellulose processing unit to at least one sugar processing unit;
(iii) from at least one sugar processing unit to at least one lignin processing unit;
(iv) from at least one sugar processing unit to at least one lignocellulose processing unit;
(v) from at least one lignin processing unit to at least one sugar processing unit; and
(vi) from at least one lignin processing unit to at least one lignocellulose processing unit.

5. A plant according to any one of claims 1 to 4, comprising a transfer mechanism transferring a material from one or more of said lignin processing units to one or more other units.

6. A plant according to any one of claims 3 to 5, comprising a transfer mechanism transferring a material from one or more of said lignin processing units to one or more of said second sugar processing units.

7. A plant according to any one of claims 3 to 6, comprising a transfer mechanism transferring a material from at least one unit of said second sugar processing units to one or more other units.

8. A plant according to any one of claims 1 to 7, wherein one or more sugar processing units is adapted to separate a sugar processing co-product from said sugar product.

9. A plant according to any one of claims 1 to 8, wherein one or more of said sugar processing units comprises one or more chemical sugar conversion modules adapted to chemically convert a sugar to a fermentable intermediate.

10. A plant according to any one of claims 1 to 9, comprising one or more conversion modules adapted to convert at least one sugar product produced by fermentation into a converted product.

11. A plant according to any one of claims 1 to 10, wherein at least one of said one or more sugar processing units comprises a recovery module adapted to recover unprocessed sugars.

12. A plant according to any one of claims 1 to 11, wherein said one or more lignocellulose processing units comprises one or more acid hydrolysis modules.

13. A plant according to any one of claims 1 to 12, comprising one or more waste water treatment units processing one or more members of the group consisting of: one or more waste streams from one or more of said lignocellulose processing units; one or more waste streams from one or more of said lignin processing units; and one or more waste streams originating from one or more of said sugar processing units.

14. A plant according to any one of claims 2 to 13, comprising one or more generators generating heat from combustion of at least one of said co-products.

15. A plant according to claim 14, comprising a heat transfer mechanism transferring heat from said generator to one or more other units.

16. A plant according to any one of claims 2 to 15, comprising a transfer mechanism transferring one or more of said lignocellulose co-products to one or more other units.

17. A plant according to any one of claims 2 to 16, wherein said lignocellulose co-product includes acetic acid and comprising an acetic acid transfer mechanism transferring acetic acid to one or more other units.

18. A plant according to any one of claims 2 to 17, wherein said lignocellulose co-product includes methanol and comprising a methanol transfer mechanism transferring methanol to one or more other units.

19. A plant according to any one of claims 2 to 18, wherein said lignocellulose co-product includes one or more tall oils and comprising a tall oil transfer mechanism transferring at least one of said one or more tall oils to one or more other units.

20. A plant according to any one of claims 2 to 19, comprising an acid transfer mechanism transferring acid from an acid recovery module of said one or more lignin processing units to one or more other units.

21. A plant according to any one of claims 1 to 20, comprising a solvent transfer mechanism transferring solvent from a solvent recovery module of said one or more lignin processing units to one or more other units.

22. A plant according to any one of claims 1 to 21, comprising one or more acid-recycle modules adapted to recover acid from one or more lignin processing units and return said acid to one or more other units.

23. A plant according to any one of claims 1 to 22, comprising a hydrogen transfer mechanism transferring hydrogen from one or more of said lignin processing units to one or more other units.

24. A plant according to any one of claims 1 to 23, wherein said lignin stream comprises liquefied lignin.

25. A plant according to any one of claims 1 to 24, wherein said one or more lignocellulose processing units process at least 10 tons of lignocellulose per hour.

26. A plant according to any one of claims 1 to 25, wherein said at least one transfer mechanism transfers at least 30,000 tons of sugar/yr from said one or more lignocellulose processing units to said one or more sugar processing units.

27. An integrated plant comprising:
(a) one or more lignocellulose processing units producing one or more sugar streams, each sugar stream including one or more sugars;
(b) one or more sugar processing units processing one or more sugars from one or more of said sugar streams into a sugar product; and
(c) at least one transfer mechanism transferring one or more of said sugar stream(s) to one or more of said sugar processing units over a distance of 5 km or less.

28. A plant according to claim 27, wherein one or more of said lignocellulose processing units includes a module which produces one or more lignocellulose co-products.

29. A plant according to claim 28, wherein said lignocellulose co-product includes acetic acid and comprising an acetic acid transfer mechanism transferring acetic acid to one or more of said sugar processing units.

30. A plant according to claim 28 or 29, wherein said lignocellulose co-product includes methanol and comprising a methanol transfer mechanism transferring methanol to one or more of said sugar processing units.

31. A plant according to any one of claims 28 to 30, wherein said lignocellulose co-product includes one or more tall oils and comprising a tall oil transfer mechanism transferring at least one of said one or more tall oils to one or more of said sugar processing units.

32. A plant according to any one of claims 28 to 31, wherein said one or more sugar processing units comprise one or more first sugar processing modules processing a first sugar into a first sugar product and one or more second sugar processing modules processing a second sugar into one or more second sugar products.

33. A plant according to claim 32, comprising at least one transfer mechanism transferring one or more of said sugar stream(s) to one or more of said second sugar processing modules.

34. A plant according to any one of claims 28 to 33, comprising at least one transfer mechanism transferring a material from one or more of said sugar processing units to one or more of said lignocellulose processing units.

35. A plant according to any one of claims 28 to 34, comprising at least one transfer mechanism transferring a sugar product to one or more sugar product processing modules.

36. A plant according to any one of claims 28 to 35, wherein said one or more lignocellulose processing units comprises one or more acid hydrolysis modules.

37. A plant according to any one of claims 28 to 36, comprising one or more waste water treatment units processing at least one member of the group consisting of:
one or more waste streams from one or more of said lignocellulose processing units; and
one or more waste streams originating from one or more of said sugar processing units.

38. A plant according to any one of claims 28 to 37, comprising one or more generators generating heat from combustion of at least one of said co-products.

39. A plant according to claim 38, comprising a heat transfer mechanism transferring heat from said generator to one unit from one or more other units.

40. A plant according to any one of claims 28 to 39, comprising a transfer mechanism transferring one or more of said lignocellulose co-products to one or more of said sugar processing units.

41. A plant according to claim 28 or claim 40, comprising:
a transfer mechanism transferring one or more sugar processing co-products from one or more of said sugar processing units to one or more other units.

42. A plant according to any one of claims 34 to 41, wherein said material includes one or more sugar processing co-products.

43. A plant according to any one of claims 34 to 42, wherein said material includes one or more sugar processing products.

44. A plant according to any one of claims 28 to 43, wherein said one or more lignocellulose processing units process at least 10 tons of lignocellulose per hour.

45. A plant according to any one of claims 28 to 44, wherein said at least one transfer mechanism transfers at least 30,000 tons of sugar/yr from said one or more lignocellulose processing units to said one or more sugar processing units.

46. An integrated plant comprising:
(a) one or more lignocellulose processing units producing at least one lignin stream;
(b) one or more lignin-processing units processing lignin from said at least one lignin stream into a lignin product; and
(c) at least one transfer mechanism transferring said lignin stream from one or more of said lignocellulose processing units to one or more of said lignin processing units over a distance of 5 km or less.

47. A plant according to claim 46, wherein one or more of said one or more lignocellulose processing units produce one or more co-products selected from the group consisting of tall oils, ash, resins, pitch and furfurals.

48. A plant according to claim 46 or claim 47, comprising a transfer mechanism transferring a material from one or more of said lignin processing units to one or more of said lignocellulose processing units.

49. A plant according to any one of claims 46 to 48, comprising an acid transfer mechanism transferring acid from an acid recovery module of said one or more lignin processing units to one or more of said lignocellulose processing units.

50. A plant according to any one of claims 46 to 49, comprising a solvent transfer mechanism transferring solvent from a solvent recovery module of said one or more lignin processing units to one or more of said lignocellulose processing units.

51. A plant according to any one of claims 46 to 50, comprising one or more acid-recycle modules adapted to recover acid from one or more lignin processing units and return said acid to said one or more of said lignocellulose processing units.

52. A plant according to any one of claims 46 to 51, comprising one or more waste-water treatment units treating at least one member of the group consisting of: one or more waste streams from one or more lignocellulose processing units and one or more waste streams from one or more lignin processing units.

53. A plant according to any one of claims 47 to 52, comprising a generator generating heat from combustion of at least one of said co-products.

54. A plant according to claim 53, comprising a heat transfer mechanism transferring heat from said generator from one unit to one or more other units.

55. A plant according to any one of claims 47 to 54, comprising a transfer mechanism transferring one or more of said co-products from one or more of said lignocellulose processing units to one or more other units.

56. A plant according to claim 52, comprising one or more transfer mechanisms transferring a waste stream from one or more of said lignin processing units to one or more of said waste-water treatment units.

57. A plant according to any one of claims 46 to 56, comprising one or more transfer mechanisms transferring a lignin processing co-product from one or more of said lignin processing units to one or more of said lignocellulose processing units.

58. A plant according to any one of claims 46 to 57, comprising one or more transfer mechanisms transferring at least a portion of said lignin product from one or more of said lignin processing units to one or more of said lignocellulose processing units.

59. A plant according to any one of claims 46 to 58, wherein said one or more lignocellulose processing units have a processing capacity of at least 10 tons of lignocellulose per hour.

60. A plant according to any one of claims 46 to 59, wherein said transfer mechanism transfers at least 10,000 tons of lignin/yr from said lignocellulose processing unit(s) to said lignin-processing unit(s).

61. An integrated plant comprising:
(a) one or more lignin-processing units processing one or more lignin streams;
(b) one or more sugar processing units processing one or more sugar streams into one or more sugar products; and
(c) at least one transfer mechanism transferring one or more materials between one or more of said sugar processing units and one or more of said lignin processing units over a distance of 5 km or less in either direction.

62. A plant according to claim 61, comprising one or more additional sugar processing units processing sugar into one or more additional sugar products.

63. A plant according to claim 62, comprising at least one transfer mechanism transferring one or more materials between one or more of said additional sugar processing units and one or more of said lignin processing units over a distance of 5 km or less in either direction.

64. A plant according to claim 62 or claim 63, comprising a transfer mechanism transferring a material from one or more of said sugar processing units to one or more of said additional sugar processing units.

65. A plant according to any one of claims 61 to 64, comprising a hydrogen transfer mechanism transferring hydrogen from one or more of said lignin processing units to one or more of said sugar processing units.

66. A plant according to claim 64 or 65, wherein said material comprises liquefied lignin.

67. A plant according to any one of claims 61 to 66, comprising a transfer mechanism transferring a material from one or more of said sugar processing units to one or more of said lignin processing units.

68. A plant according to claim 67, wherein said material includes a solvent produced by an Aqueous phase reforming (APR) module in one or more of said sugar processing units.

69. A plant according to claim 67 or 68, wherein said material includes an alcohol.

70. A plant according to any one of claims 62 to 69, comprising a transfer mechanism transferring a material from one or more of said lignin processing units to one or more of said additional sugar processing units.

71. A plant according to claim 70, wherein said material comprises hydrogen.

72. A plant according to claim 70 or 71, wherein said material comprises liquefied lignin.

73. A plant according any one of claims 61 to 72, comprising a transfer mechanism transferring a material from at least one unit of said sugar processing units into one or more of said lignin processing units.

74. A plant according to any one of claims 61 to 73, comprising one or more waste water treatment units processing at least one stream selected from the group consisting of: one or more waste streams from one or more of said lignin processing units; and one or more waste streams originating from one or more of said sugar processing units.

75. A plant according to any one of claims 61 to 74, comprising one or more generators generating heat from combustion of at least one of said co-products.

76. A plant according to claim 75, comprising a heat transfer mechanism transferring heat from said generator to one or more other units.

77. A plant according to any one of claims 61 to 76, wherein said one or more sugar processing units have a processing capacity of at least 3 tons of sugar per hour.
